(19) United States
(12) Patent Application Publication
(10) Pub. No.: US 2022/0072049 A1
(43) Pub. Date: Mar. 10, 2022

(54) COMPOSITIONS AND METHODS OF TREATMENT USING MICROVESICLES FROM BONE MARROW-DERIVED MESENCHYMAL STEM CELLS

(71) Applicant: UNIVERSITY OF MIAMI, Miami, FL (US)

(72) Inventor: Evangelos V. Badiavas, Coral Gables, FL (US)

(21) Appl. No.: 17/407,682

(22) Filed: Aug. 20, 2021

Related U.S. Application Data
(60) Provisional application No. 63/068,517, filed on Aug. 21, 2020.

(51) Int. Cl. A61K 35/28; C12N 5/0775

(52) U.S. Cl. CPC A61K 35/28; C12N 5/0663

(57) ABSTRACT

Methods for the treatment of a variety of conditions using microvesicles from bone marrow-derived mesenchymal stem cells are described.

### CROSS-REFERENCE TO RELATED APPLICATION

This application claims priority to pending U.S. Provisional Patent Application Ser. No. 63/068,517, filed Aug. 21, 2021, the entire content of which is incorporated by reference in its entirety herein.

### FIELD OF THE INVENTION

The present invention relates to the fields of medicine, cell biology, molecular biology and genetics. In particular, the present invention relates to compositions and methods for treating various medical conditions using microvesicles from bone marrow-derived mesenchymal stem cells.

### BACKGROUND

The relationship between the skin and other body tissues, such as the bone marrow, is complex and relies on interaction and exchange of information and signals, including secreted proteins. The bone marrow serves key roles in maintaining skin homeostasis. The relationship of bone marrow to skin is intricately connected via its secretome—the totality of proteins produced by the bone marrow that can serve functions in skin tissues.

In patients that have dysfunctional bone marrow, the skin may be the first sign of an underlying pathology through, for example, development of chronic wounds, changes in pigmentation, and infection. (See Badiavas E V, Ford D, Liu P, Kouttab N, Morgan J, Richards A et al. Long-term bone marrow culture and its clinical potential in chronic wound healing. Wound repair and regeneration: official publication of the Wound Healing Society [and] the European Tissue Repair Society 2007; 15:856-65). In subjects with genetic mutations resulting in dermatologic phenotypes, such as forms of epidermolysis bullosa, bone marrow transplants have been shown to be effective in attenuating skin pathology. (See Wagner J E, Ishida-Yamamoto A, McGrath J A, Hordinsky M, Keene D R, Woodley D T et al. Bone marrow transplantation for recessive dystrophic epidermolysis bullosa. N Engl J Med 2010; 363:629-39). Because bone marrow-derived mesenchymal cells (BM-MSCs) have been shown to be beneficial in a variety of diseases, included wound healing, but engraftment and survival into other tissues after transplant is very low, the exact mechanisms as to how patients experience benefit from cellular therapy remains to be fully understood. (See Isakson M, de Blacam C, Whelan D, McArdle A, Clover A J. Mesenchymal Stem Cells and Cutaneous Wound Healing: Current Evidence and Future Potential. Stem Cells Int 2015:831095; Badiavas E V, Falanga V. Treatment of chronic wounds with bone marrow-derived cells. Arch Dermatol 2003; 139:510-6; and Dash N R, Dash S N, Routray P, Mohapatra S, Mohapatra P C. Targeting nonhealing ulcers of lower extremity in human through autologous bone marrow-derived mesenchymal stem cells. Rejuvenation Res 2009; 12:359-66). Treatment methods of these various diseases are greatly expanded if the beneficial effects are mediated through the bone marrow cells' secretome, independent of direct cell-engraftment into the skin.

### SUMMARY

The disclosure provides compositions and methods for treating various medical conditions using microvesicles from bone marrow-derived mesenchymal stem cells.

The disclosure provides, in one aspect, a method of treating a condition selected from the group consisting of epidermolysis bullosa pruriginosa; epidermolysis bullosa acquisita; epidermolysis bullosa dystrophica, pretibial type; epidermolysis bullosa dystrophica, bart type; nonsyndromic congenital nail disorder-8; epidermolysis bullosa dystrophica, with subcorneal cleavage; and transient bullous dermolysis of the newborn in a subject in need thereof comprising administering a therapeutically effective amount of microvesicles, wherein the microvesicles comprise type VII collagen. In some embodiments, the subject has a mutation in the COL7A1 gene. In some embodiments, the microvesicles deliver collagen VII protein to the cells of the subject.

In some embodiments, the condition is epidermolysis bullosa pruriginosa. In some embodiments, the microvesicles alleviate or reduce one or more symptoms of epidermolysis bullosa pruriginosa in the subject. In some embodiments, the symptoms of epidermolysis bullosa pruriginosa are selected from the group consisting of pruritus, blisters, chronic wounds, scar formation, increased risk of skin infections, milia, skin fragility, nail dystrophy, lichenified plaques, albopapuloid lesions, and excoriated prurigo nodules.

In some embodiments, the condition is epidermolysis bullosa acquisita. In some embodiments, the microvesicles alleviate or reduce one or more symptoms of epidermolysis bullosa acquisita in the subject. In some embodiments, the symptoms of epidermolysis bullosa acquisita are selected from the group consisting of blistering, milia, wound healing with significant scarring, skin itching, and skin redness.

In some embodiments, the condition is epidermolysis bullosa dystrophica, pretibial type. In some embodiments, the microvesicles alleviate or reduce one or more symptoms of epidermolysis bullosa dystrophica, pretibial type in the subject. In some embodiments, the symptoms of epidermolysis bullosa dystrophica, pretibial type are selected from the group consisting of pretibial blisters, prurigo-like hyperkeratotic lesions, nail dystrophy, albopapuloid skin lesions, and hypertrophic scars.

In some embodiments, the condition is epidermolysis bullosa dystrophica, bart type. In some embodiments, the microvesicles alleviate or reduce one or more symptoms of epidermolysis bullosa dystrophica, bart type in the subject. In some embodiments, the symptoms of epidermolysis bullosa dystrophica, bart type are selected from the group consisting of congenital localized absence of skin, skin fragility, and deformity of the nails.

In some embodiments, the condition is nonsyndromic congenital nail disorder-8. In some embodiments, the microvesicles alleviate or reduce one or more symptoms of nonsyndromic congenital nail disorder-8 in the subject. In some embodiments, the symptoms of nonsyndromic congenital nail disorder-8 comprise toenail dystrophy and/or the nail plate being buried in the nail bed with a deformed and narrow free edge.

In some embodiments, the condition is epidermolysis bullosa dystrophica, with subcorneal cleavage. In some embodiments, the microvesicles alleviate or reduce one or more symptoms of epidermolysis bullosa dystrophica, with subcorneal cleavage in the subject. In some embodiments, the symptoms of epidermolysis bullosa dystrophica, with subcorneal cleavage are selected from the group consisting of blisters, milia, atrophic scarring, and nail dystrophy.

In some embodiments, the condition is transient bullous dermolysis of the newborn. In some embodiments, the microvesicles alleviate or reduce one or more symptoms of transient bullous dermolysis of the newborn in the subject. In some embodiments, the symptoms of transient bullous dermolysis of the newborn are selected from the group consisting of sub-epidermal blisters, reduced or abnormal anchoring fibrils at the dermo-epidermal junction, and electron-dense inclusions in keratinocytes.

In another aspect, the disclosure provides a method of treating Alport syndrome 2, autosomal recessive in a subject in need thereof comprising administering a therapeutically effective amount of microvesicles, wherein the microvesicles comprise type IV collagen. In some embodiments, the microvesicles alleviate or reduce one or more symptoms of Alport syndrome 2, autosomal recessive in the subject. In some embodiments, the symptoms of Alport syndrome 2, autosomal recessive are selected from the group consisting of glomerulonephritis, glomerular basement membrane defects, renal failure, sensorineural deafness, lenticonous, macular flecks, and hematuria. In some embodiments, the subject has a mutation in the COL4A4 gene. In some embodiments, the microvesicles deliver type IV collagen protein to the cells of the subject.

In yet another aspect, the disclosure provides a method of treating a condition selected from the group consisting of epidermolysis bullosa simplex with muscular dystrophy; epidermolysis bullosa simplex with pyloric atresia; epidermolysis bullosa, ogna type; epidermolysis bullosa simplex with nail dystrophy; and muscular dystrophy, limb-girdle, autosomal recessive 17 in a subject in need thereof comprising administering a therapeutically effective amount of microvesicles, wherein the microvesicles comprise plectin. In some embodiments, the subject has a mutation in the PLEC1 gene. In some embodiments, the microvesicles deliver plectin protein to the cells of the subject.

In some embodiments, the condition is epidermolysis bullosa simplex with muscular dystrophy. In some embodiments, the microvesicles alleviate or reduce one or more symptoms of epidermolysis bullosa simplex with muscular dystrophy in the subject. In some embodiments, the symptoms of epidermolysis bullosa simplex with muscular dystrophy are selected from the group consisting of hemorrhagic blisters, blister formation at the level of the hemidesmosome, nail dystrophy, palmoplantar keratoderma, and erosions of the skin and oral mucosae.

In some embodiments, the condition is epidermolysis bullosa simplex with pyloric atresia. In some embodiments, the microvesicles alleviate or reduce one or more symptoms of epidermolysis bullosa simplex with pyloric atresia in the subject. In some embodiments, the symptoms of epidermolysis bullosa simplex with pyloric atresia are selected from the group consisting of blistering, skin fragility, milia, nail dystrophy, scarring alopecia, and hypotrichosis.

In some embodiments, the condition is epidermolysis bullosa, ogna type. In some embodiments, the microvesicles alleviate or reduce one or more symptoms of epidermolysis bullosa, ogna type in the subject. In some embodiments, the symptoms of epidermolysis bullosa, ogna type are selected from the group consisting of skin bruising, skin fragility, blistering, and abnormal hemidesmosome intracellular attachment plates.

In some embodiments, the condition is epidermolysis bullosa simplex with nail dystrophy. In some embodiments, the microvesicles alleviate or reduce one or more symptoms of epidermolysis bullosa simplex with nail dystrophy in the subject. In some embodiments, the symptoms of epidermolysis bullosa simplex with nail dystrophy comprise skin blistering and/or nail dystrophy.

In some embodiments, the condition is muscular dystrophy, limb-girdle, autosomal recessive 17. In some embodiments, the microvesicles alleviate or reduce one or more symptoms of muscular dystrophy, limb-girdle, autosomal recessive 17 in the subject. In some embodiments, the symptoms of muscular dystrophy, limb-girdle, autosomal recessive 17 are selected from the group consisting of proximal muscle weakness, weakness of the hip and shoulder girdles, prominent asymmetrical quadriceps femoris atrophy, and biceps brachii atrophy.

The disclosure provides, in one aspect, a method of treating a condition selected from the group consisting of epidermolysis bullosa simplex, autosomal recessive 2 and neuropathy, hereditary sensory and autonomic, 6 in a subject in need thereof comprising administering a therapeutically effective amount of microvesicles, wherein the microvesicles comprise bullous pemphigoid antigen 1. In some embodiments, the subject has a mutation in the BPAG1 gene.

In some embodiments, the microvesicles deliver bullous pemphigoid antigen 1 protein to the cells of the subject.

In some embodiments, the condition is epidermolysis bullosa simplex, autosomal recessive 2. In some embodiments, the microvesicles alleviate or reduce one or more symptoms of epidermolysis bullosa simplex, autosomal recessive 2 in the subject. In some embodiments, the symptoms of epidermolysis bullosa simplex, autosomal recessive 2 are selected from the group consisting of blistering on the dorsal, lateral and plantar surfaces of the feet, trauma-induced blistering on the feet and ankles, and abnormal hemidesmosomes with poorly formed inner plaques.

In some embodiments, the condition is neuropathy, hereditary sensory and autonomic, 6. In some embodiments, the microvesicles alleviate or reduce one or more symptoms of neuropathy, hereditary sensory and autonomic, 6 in the subject. In some embodiments, the symptoms of neuropathy, hereditary sensory and autonomic, 6 are selected from the group consisting of degeneration of dorsal root and autonomic ganglion cells, sensory abnormalities, and autonomic abnormalities.

In another aspect, the disclosure provides a method of treating epidermolytic hyperkeratosis in a subject in need thereof comprising administering a therapeutically effective amount of microvesicles, wherein the microvesicles comprise keratin 1. In some embodiments, the microvesicles alleviate or reduce one or more symptoms of epidermolytic hyperkeratosis in the subject. In some embodiments, the symptoms of epidermolytic hyperkeratosis are selected from the group consisting of intraepidermal blistering, thickening of the stratum corneum, pigmentation of the skin and erosions at sites of trauma, and erythroderma. In some embodiments, the subject has a mutation in the KRT1 gene. In some embodiments, the microvesicles deliver keratin 1 protein to the cells of the subject.

In yet another aspect, the disclosure provides a method of treating benign familial pemphigus in a subject in need thereof comprising administering a therapeutically effective amount of microvesicles, wherein the microvesicles comprise hSPCA1. In some embodiments, the microvesicles alleviate or reduce one or more symptoms of benign familial pemphigus in the subject. In some embodiments, the symptoms of benign familial pemphigus are selected from the group consisting of blisters, erosions of the skin, rash, cracked skin, and acantholysis. In some embodiments, the subject has a mutation in the ATP2C1 gene. In some embodiments, the microvesicles deliver hSPCA1 protein to the cells of the subject.

In another aspect, the disclosure provides method of treating Chediak-Higashi syndrome in a subject in need thereof comprising administering a therapeutically effective amount of microvesicles, wherein the microvesicles comprise lysosomal trafficking regulator. In some embodiments, the microvesicles alleviate or reduce one or more symptoms of Chediak-Higashi syndrome in the subject. In some embodiments, the symptoms of Chediak-Higashi syndrome are selected from the group consisting of hypopigmentation, severe immunologic deficiency, bleeding tendency, neurologic abnormalities, abnormal intracellular transport to and from the lysosome, and giant inclusion bodies in a variety of cell types. In some embodiments, the subject has a mutation in the LYST gene. In some embodiments, the microvesicles deliver lysosomal trafficking regulator protein to the cells of the subject.

In still another aspect, a method of treating a condition selected from the group consisting of ataxia telangiectasia syndrome; T-cell acute lymphoblastic leukemia; and B-cell chronic lymphocytic leukemia in a subject in need thereof comprising administering a therapeutically effective amount of microvesicles, wherein the microvesicles comprise serine-protein kinase ATM. In some embodiments, the subject has a mutation in the ATM gene. In some embodiments, the microvesicles deliver serine-protein kinase ATM protein to the cells of the subject.

In some embodiments, the condition is ataxia telangiectasia syndrome. In some embodiments, the microvesicles alleviate or reduce one or more symptoms of ataxia telangiectasia syndrome in the subject. In some embodiments, the symptoms of ataxia telangiectasia syndrome are selected from the group consisting of progressive cerebellar ataxia, dilation of the blood vessels in the conjunctiva and eyeballs, immunodeficiency, growth retardation, and sexual immaturity.

In some embodiments, the condition is T-cell acute lymphoblastic leukemia. In some embodiments, the microvesicles alleviate or reduce one or more symptoms of T-cell acute lymphoblastic leukemia in the subject. In some embodiments, the symptoms of T-cell acute lymphoblastic leukemia are selected from the group consisting of anemia, frequent infections due to the lack of normal white blood cells, frequent infections, fever, purpura, and nosebleeds and bleeding gums due to lack of platelets.

In some embodiments, the condition is T-cell prolymphocytic leukemia. In some embodiments, the microvesicles alleviate or reduce one or more symptoms of T-cell prolymphocytic leukemia. In some embodiments, the symptoms of T-cell prolymphocytic leukemia are selected from the group consisting of a high white blood cell count, a predominance of prolymphocytes, marked splenomegaly, lymphadenopathy, skin lesions, and serous effusion.

In some embodiments, the condition is B-cell chronic lymphocytic leukemia. In some embodiments, the microvesicles alleviate or reduce one or more symptoms of B-cell chronic lymphocytic leukemia in the subject. In some embodiments, the symptoms of B-cell chronic lymphocytic leukemia are selected from the group consisting of accumulation of mature CD5+B-lymphocytes, lymphadenopathy, immunodeficiency, and bone marrow failure.

In another aspect, the disclosure provides a method of treating tuberous sclerosis 2 in a subject in need thereof comprising administering a therapeutically effective amount of microvesicles, wherein the microvesicles comprise tuberin. In some embodiments, the microvesicles alleviate or reduce one or more symptoms of tuberous sclerosis 2 in the subject. In some embodiments, the symptoms of tuberous sclerosis 2 are selected from the group consisting of hamartomas, hamartias, epilepsy, learning difficulties, behavioral problems, and skin lesions. In some embodiments, the subject has a mutation in the TSC2 gene. In some embodiments, the microvesicles deliver tuberin protein to the cells of the subject.

In still another aspect, the disclosure provides method of treating diabetic foot ulcers in a subject in need thereof comprising administering a therapeutically effective amount of microvesicles, wherein the microvesicles comprise FOXM1A. In some embodiments, the microvesicles alleviate or reduce one or more symptoms of diabetic foot ulcers in the subject. In some embodiments, the symptoms of diabetic foot ulcers comprise open sores or wounds on the foot of the subject. In some embodiments, the subject has a mutation in the FOXM1A gene. In some embodiments, the microvesicles deliver FOXM1A protein to the cells of the subject.

In another aspect, the disclosure provides a method of treatment, wherein the microvesicles are derived from mesenchymal stem cells. In some embodiments, the mesenchymal stem cells are bone marrow mesenchymal stem cells.

In yet another aspect, the disclosure provides a method of treatment wherein the microvesicles are obtained from a biological fluid and precipitated from the biological fluid using polyethylene glycol.

In still another aspect, the disclosure provides a method of treatment wherein the microvesicles are administered to the skin and/or nails of the subject.

In another aspect, the disclosure provides a method of treatment wherein the microvesicles are administered via transplanted mesenchymal stem cells.

### BRIEF DESCRIPTION OF THE DRAWINGS

FIG. 1 depicts a flow chart of the study design of Example 1 wherein unique proteins were identified from the bone marrow-derived mesenchymal cells (BM-MSC) secretome of four bone marrow donors.

FIG. 2 graphically depicts the number of proteins obtained from the BM-MSC secretome of the four bone marrow donors of Example 1 classified by cellular components.

FIG. 3 graphically depicts the number of proteins obtained from the BM-MSC secretome of the four bone marrow donors of Example 1 classified by biological processes.

FIG. 4 graphically depicts the number of proteins obtained from the BM-MSC secretome of the four bone marrow donors of Example 1 classified by ligand functions.

FIG. 5 graphically depicts the number of proteins obtained from the BM-MSC secretome of the four bone marrow donors of Example 1 classified by molecular functions.

FIG. 6 graphically depicts the number of proteins obtained from the BM-MSC secretome of the four bone marrow donors of Example 1 classified by disease correlations.

FIG. 7 shows one embodiment of an apparatus described herein that facilitates the clarification of the biological fluid and the collection of the precipitated microvesicles by filtration.

### DETAILED DESCRIPTION

Before the invention is described, it is to be understood that this invention is not limited to particular methods and experimental conditions described, as such methods and conditions may vary. It is also to be understood that the terminology used herein is for the purpose of describing particular embodiments only, and is not intended to be limiting, because the scope of the invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition. In some embodiments, a subject to be treated is selected based on the presence of symptoms of a disorder or condition. In some embodiments, a subject is first diagnosed with a disorder or condition and is then treated for that disorder or condition. In some embodiments, the disorder or condition is one or more of those described below.

Although any methods and materials similar or equivalent to those described herein can be used in the practice of the invention, the typical methods and materials are now described. All publications mentioned herein are incorporated herein by reference in their entirety.

### Methods for Treating Type VII Collagen Related Conditions

In some embodiments, the invention provides methods for treating epidermolysis bullosa pruriginosa; epidermolysis bullosa acquisita; epidermolysis bullosa dystrophica, pretibial type; epidermolysis bullosa dystrophica, bart type; nonsyndromic congenital nail disorder-8; epidermolysis bullosa dystrophica, with subcorneal cleavage; or transient bullous dermolysis of the newborn, wherein the methods comprise administering a therapeutically effective amount of microvesicles to the subject.

In some embodiments, the subject has a mutation in the COL7A1 gene.

In some embodiments, the microvesicles deliver collagen VII protein to the cells of the subject. Type VII collagen is present in the stratified squamous epithelial basement membrane and forms the anchoring fibrils that contribute to epithelial basement membrane organization and adherence by interacting with extracellular matrix proteins, such as type IV collagen.

### Epidermolysis Bullosa Pruriginosa

In some embodiments, the invention encompasses methods to treat or alleviate conditions or complications associated with epidermolysis bullosa pruriginosa. Epidermolysis bullosa pruriginosa, also referred to as EB pruriginosa, is clinically heterogeneous subtype of dystrophic epidermolysis bullosa resulting from a mutation within the type VII collagen gene. Due to the absence of collagen VII in the skin, patients with epidermolysis bullosa pruriginosa develop severe blistering, resulting in widespread chronic wounds, scarring and increased risk of infections. Onset is in early childhood, but in some cases is delayed until the second or third decade of life. Inheritance can be autosomal dominant or recessive. Epidermolysis bullosa pruriginosa is associated with mutations of the COL7A1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa pruriginosa, or who has been diagnosed with epidermolysis bullosa pruriginosa.

In some embodiments, microvesicles are administered to a subject in need thereof to alleviate the symptoms or complications associated with epidermolysis bullosa pruriginosa. In some embodiments the symptoms associated with epidermolysis bullosa pruriginosa include pruritus, blisters, chronic wounds, scar formation, increased risk of skin infections, milia, skin fragility, nail dystrophy, lichenified plaques, albopapuloid lesions, and excoriated prurigo nodules.

### Epidermolysis Bullosa Acquisita

In some embodiments, the invention encompasses methods to treat or alleviate conditions or complications associated with epidermolysis bullosa acquisita. Epidermolysis bullosa acquisita (EBA) is an autoimmune acquired blistering skin disease resulting from autoantibodies to type VII collagen. This rare autoimmune disease is characterized by sub-epithelial blistering of the skin and mucosal membranes in response to injury. Blisters associated with epidermolysis bullosa acquisita tend to be localized to areas that are easily injured such as the hands, feet, knees, elbows, and buttocks.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa acquisita, or who has been diagnosed with epidermolysis bullosa acquisita.

In some embodiments, microvesicles are administered to a subject in need thereof to alleviate the symptoms or complications associated with epidermolysis bullosa acquisita. In some embodiments the symptoms associated with epidermolysis bullosa acquisita include blistering, milia, wound healing with significant scarring, skin itching, and skin redness.

### Epidermolysis Bullosa Dystrophica, Pretibial Type

In some embodiments, the invention encompasses methods to treat or alleviate conditions or complications associated with epidermolysis bullosa dystrophica, pretibial type. Epidermolysis bullosa dystrophica, pretibial type (PR-DEB) is a form of dystrophic epidermolysis bullosa characterized by pretibial blisters that develop into prurigo-like hyperkeratotic lesions. It predominantly affects the pretibial areas, sparing the knees and other parts of the skin. Other clinical features include nail dystrophy, albopapuloid skin lesions, and hypertrophic scars without pretibial predominance. The phenotype shows considerable interindividual variability. Inheritance is autosomal dominant. Epidermolysis bullosa dystrophica, pretibial type is associated with mutations of the COL7A1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa dystrophica, pretibial type, or who has been diagnosed with epidermolysis bullosa dystrophica, pretibial type.

In some embodiments, microvesicles are administered to a subject in need thereof to alleviate the symptoms or complications associated with epidermolysis bullosa dystrophica, pretibial type. In some embodiments the symptoms associated with epidermolysis bullosa dystrophica, pretibial type include pretibial blisters, prurigo-like hyperkeratotic lesions, nail dystrophy, albopapuloid skin lesions, and hypertrophic scars.

### Epidermolysis Bullosa Dystrophica, Bart Type

In some embodiments, the invention encompasses methods to treat or alleviate conditions or complications associated with epidermolysis bullosa dystrophica, bart type. Epidermolysis bullosa dystrophica, bart type (B-DEB) is an autosomal dominant form of dystrophic epidermolysis bullosa characterized by congenital localized absence of skin, skin fragility and deformity of nails. Epidermolysis bullosa dystrophica, bart type is associated with mutations of the COL7A1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa dystrophica, bart type, or who has been diagnosed with epidermolysis bullosa dystrophica, bart type.

In some embodiments, microvesicles are administered to a subject in need thereof to alleviate the symptoms or complications associated with epidermolysis bullosa dystrophica, bart type. In some embodiments the symptoms associated with epidermolysis bullosa dystrophica, bart type include congenital localized absence of skin, skin fragility, and deformity of the nails.

### Nonsyndromic Congenital Nail Disorder-8

In some embodiments, the invention encompasses methods to treat or alleviate conditions or complications associated with nonsyndromic congenital nail disorder-8. Nail disorder, non-syndromic congenital, 8 (NDNC8) is a nail disorder characterized by isolated toenail dystrophy. The nail changes are most severe in the great toes and consist of the nail plate being buried in the nail bed with a deformed and narrow free edge. This form of isolated toenail dystrophy has been found to segregate as an autosomal dominant trait in families in which another member has the autosomal recessive skin disorder dystrophic epidermolysis bullosa or transient bullous dermolysis of the newborn. Nail disorder, non-syndromic congenital, 8 is associated with mutations of the COL7A1 gene.

The methods featured in the invention include administering to a subject in need thereof a therapeutic composition comprising microvesicles. In some embodiments “a subject in need thereof” means a human or non-human animal that exhibits one or more symptoms or indicia of nonsyndromic congenital nail disorder-8, or who has been diagnosed with nonsyndromic congenital nail disorder-8.

In some embodiments, microvesicles are administered to a subject in need thereof to alleviate the symptoms or complications associated with nonsyndromic congenital nail disorder-8. In some embodiments the symptoms associated with nonsyndromic congenital nail disorder-8 include toenail dystrophy and the nail plate being buried in the nail bed with a deformed and narrow free edge.

Epidermolysis Bullosa Dystrophica, with Subcorneal Cleavage

In some embodiments, the invention encompasses methods to treat or alleviate conditions or complications associated with epidermolysis bullosa dystrophica, with subcorneal cleavage. Epidermolysis bullosa dystrophica, with subcorneal cleavage is a bullous skin disorder with variable sized clefts just beneath the level of the stratum corneum. Epidermolysis bullosa dystrophica, with subcorneal cleavage is associated with mutations of the COL7A/gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa dystrophica, with subcorneal cleavage, or who has been diagnosed with epidermolysis bullosa dystrophica, with subcorneal cleavage.

In some embodiments, microvesicles are administered to a subject in need thereof to alleviate the symptoms or complications associated with epidermolysis bullosa dystrophica, with subcorneal cleavage. In some embodiments the symptoms associated with epidermolysis bullosa dystrophica, with subcorneal cleavage include blisters, milia, atrophic scarring, and nail dystrophy.

### Transient Bullous Dermolysis of the Newborn

In some embodiments, the invention encompasses methods to treat or alleviate conditions or complications associated with transient bullous dermolysis of the newborn. Transient bullous dermolysis of the newborn (TBDN) is a neonatal form of dystrophic epidermolysis bullosa that is characterized by blister formation as a result of even mild trauma. Transient bullous dermolysis of the newborn is an inherited condition associated with COL7A1.

The methods featured in the invention include administering to a subject in need thereof a therapeutic composition comprising microvesicles. In some embodiments “a subject in need thereof” means a human or non-human animal that exhibits one or more symptoms or indicia of transient bullous dermolysis of the newborn, or who has been diagnosed with transient bullous dermolysis of the newborn.

In some embodiments, microvesicles are administered to a subject in need thereof to alleviate the symptoms or complications associated with transient bullous dermolysis of the newborn. In some embodiments the symptoms associated with transient bullous dermolysis of the newborn include sub-epidermal blisters, reduced or abnormal anchoring fibrils at the dermo-epidermal junction, and electron-dense inclusions in keratinocytes.

### Methods for Treating Type IV Collagen Related Conditions

In some embodiments, the invention provides methods for treating Alport syndrome 2, autosomal recessive, wherein the methods comprise administering a therapeutically effective amount of microvesicles to the subject.

In some embodiments, the subject has a mutation in the COL4A4 gene.

In some embodiments, the microvesicles deliver type IV collagen protein to the cells of the subject. Type IV collagen is the major structural component of the cutaneous and glomerular basement membrane, it forms a meshwork together with laminins, proteoglycans and entactin/nidogen.

### Alport Syndrome 2, Autosomal Recessive

In some embodiments, the invention encompasses methods to treat or alleviate conditions or complications associated with Alport syndrome 2, autosomal recessive. Alport syndrome 2, autosomal recessive is a syndrome characterized by progressive glomerulonephritis, glomerular basement membrane defects, renal failure, sensorineural deafness and specific eye abnormalities (lenticonous and macular flecks). The disorder shows considerable heterogeneity in that families differ in the age of end-stage renal disease and the occurrence of deafness. Loss of protein can result in benign familial hematuria. Alport syndrome 2, autosomal recessive is characterized by non-progressive isolated microscopic hematuria that does not result in renal failure. It is characterized pathologically by thinning of the glomerular basement membrane. Alport syndrome 2, autosomal recessive is associated with mutations of the COL4A4 gene.

The methods featured in the invention include administering to a subject in need thereof a therapeutic composition comprising microvesicles. In some embodiments “a subject in need thereof” means a human or non-human animal that exhibits one or more symptoms or indicia of Alport syndrome 2, autosomal recessive, or who has been diagnosed with Alport syndrome 2, autosomal recessive.

In some embodiments, microvesicles are administered to a subject in need thereof to alleviate the symptoms or complications associated with Alport syndrome 2, autosomal recessive. In some embodiments the symptoms associated with Alport syndrome 2, autosomal recessive include glomerulonephritis, glomerular basement membrane defects, renal failure, sensorineural deafness, lenticonous, macular flecks, and hematuria.

### Methods for Treating Plectin Related Conditions

In some embodiments, the invention provides methods for treating epidermolysis bullosa simplex with muscular dystrophy; epidermolysis bullosa simplex with pyloric atresia; epidermolysis bullosa, ogna type; epidermolysis bullosa simplex with nail dystrophy; or muscular dystrophy, limb-girdle, autosomal recessive 17, wherein the methods comprise administering a therapeutically effective amount of microvesicles to the subject.

In some embodiments, the subject has a mutation in the PLEC1 gene.

In some embodiments, the microvesicles deliver plectin protein to the cells of the subject. Plectin is also referred to as PCN, PLTN, hemidesmosomal protein 1, HD1, and plectin-1. Plectin interlinks intermediate filaments with microtubules and microfilaments and also anchors intermediate filaments to desmosomes or hemidesmosomes. Plectin binds muscle proteins such as actin to membrane complexes in muscle. Plectin also plays a major role in the maintenance of myofiber integrity.

Epidermolysis Bullosa Simplex with Muscular Dystrophy

In some embodiments, the invention encompasses methods to treat or alleviate conditions or complications associated with epidermolysis bullosa simplex with muscular dystrophy. Epidermolysis bullosa simplex, with muscular dystrophy (MD-EBS) is a form of epidermolysis bullosa characterized by the association of blister formation at the level of the hemidesmosome and late-onset muscular dystrophy. Epidermolysis bullosa simplex with muscular dystrophy is a rare life-threatening subtype of basal Epidermolysis bullosa simplex with autosomal recessive inheritance. Epidermolysis bullosa simplex with muscular dystrophy is associated with mutations of the PLEC1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa simplex with muscular dystrophy, or who has been diagnosed with epidermolysis bullosa simplex with muscular dystrophy.

In some embodiments, microvesicles are administered to a subject in need thereof to alleviate the symptoms or complications associated with epidermolysis bullosa simplex with muscular dystrophy. In some embodiments the symptoms associated with epidermolysis bullosa simplex with muscular dystrophy include hemorrhagic blisters, blister formation at the level of the hemidesmosome, nail dystrophy, palmoplantar keratoderma, and erosions of the skin and oral mucosae.

Epidermolysis Bullosa Simplex with Pyloric Atresia

In some embodiments, the invention encompasses methods to treat or alleviate conditions or complications associated with epidermolysis bullosa simplex with pyloric atresia. Epidermolysis bullosa simplex with pyloric atresia is an autosomal recessive genodermatosis characterized by severe skin blistering at birth and congenital pyloric atresia. Death usually occurs in infancy. Epidermolysis bullosa simplex with pyloric atresia is associated with mutations of the PLEC1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa simplex with pyloric atresia, or who has been diagnosed with epidermolysis bullosa simplex with pyloric atresia.

In some embodiments, microvesicles are administered to a subject in need thereof to alleviate the symptoms or complications associated with epidermolysis bullosa simplex with pyloric atresia. In some embodiments the symptoms associated with epidermolysis bullosa simplex with pyloric atresia include blistering, skin fragility, milia, nail dystrophy, scarring alopecia, and hypotrichosis.

### Epidermolysis Bullosa, Ogna Type

In some embodiments, the invention encompasses methods to treat or alleviate conditions or complications associated with epidermolysis bullosa, ogna type. Epidermolysis bullosa simplex, ogna type (0-EBS) is a form of intraepidermal epidermolysis bullosa characterized by generalized skin bruising, skin fragility with non-scarring blistering and small hemorrhagic blisters on the hands. At the ultrastructural level, it is differentiated from other varieties of epidermolysis bullosa by the occurrence of blisters originating in basal cells above hemidesmosomes, and abnormal hemidesmosome intracellular attachment plates. Epidermolysis bullosa, ogna type is associated with mutations of the PLEC1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa, ogna type, or who has been diagnosed with epidermolysis bullosa, ogna type.

In some embodiments, microvesicles are administered to a subject in need thereof to alleviate the symptoms or complications associated with epidermolysis bullosa, ogna type. In some embodiments the symptoms associated with epidermolysis bullosa, ogna type include skin bruising, skin fragility, blistering, and abnormal hemidesmosome intracellular attachment plates.

Epidermolysis Bullosa Simplex with Nail Dystrophy

In some embodiments, the invention encompasses methods to treat or alleviate conditions or complications associated with epidermolysis bullosa simplex with nail dystrophy. Epidermolysis bullosa simplex with nail dystrophy (EBSND) is a form of epidermolysis bullosa, a dermatologic disorder characterized by skin blistering and nail dystrophy. Inheritance is autosomal recessive and onset is in childhood with exacerbation during puberty. Epidermolysis bullosa simplex with nail dystrophy is associated with mutations of the PLEC1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa simplex with nail dystrophy, or who has been diagnosed with epidermolysis bullosa simplex with nail dystrophy.

In some embodiments, microvesicles are administered to a subject in need thereof to alleviate the symptoms or complications associated with epidermolysis bullosa simplex with nail dystrophy. In some embodiments the symptoms associated with epidermolysis bullosa simplex with nail dystrophy include skin blistering and nail dystrophy.

### Muscular Dystrophy, Limb-Girdle, Autosomal Recessive 17

In some embodiments, the invention encompasses methods to treat or alleviate conditions or complications associated with muscular dystrophy, limb-girdle, autosomal recessive 17. Muscular dystrophy, limb-girdle, autosomal recessive 17 is a form of limb-girdle muscular dystrophy characterized by early childhood onset of proximal muscle weakness and atrophy without skin involvement. Muscular dystrophy, limb-girdle, autosomal recessive 17 is associated with mutations of the PLEC1 gene.

The methods featured in the invention include administering to a subject in need thereof a therapeutic composition comprising microvesicles. In some embodiments “a subject in need thereof” means a human or non-human animal that exhibits one or more symptoms or indicia of muscular dystrophy, limb-girdle, autosomal recessive 17, or who has been diagnosed with muscular dystrophy, limb-girdle, autosomal recessive 17.

In some embodiments, microvesicles are administered to a subject in need thereof to alleviate the symptoms or complications associated with muscular dystrophy, limb-girdle, autosomal recessive 17. In some embodiments the symptoms associated with muscular dystrophy, limb-girdle, autosomal recessive 17 include proximal muscle weakness, weakness of the hip and shoulder girdles, prominent asymmetrical quadriceps femoris atrophy, and biceps brachii atrophy.

### Methods for Treating Bullous Pemphigoid Antigen 1 Related Conditions

In some embodiments, the invention provides methods for treating epidermolysis bullosa simplex, autosomal recessive 2 or neuropathy, hereditary sensory and autonomic, 6, wherein the methods comprise administering a therapeutically effective amount of microvesicles to the subject.

In some embodiments, the subject has a mutation in the BPAG1 gene, also known as DST, and BP230.

In some embodiments, the microvesicles deliver bullous pemphigoid antigen 1 protein to the cells of the subject. Bullous pemphigoid antigen 1 is also known as dystonin, BPA (Bullous pemphigoid antigen), dystonia musculorum protein, and hemidesmosomal plaque protein. Bullous pemphigoid antigen 1 is a cytoskeletal linker protein that acts as a connector between intermediate filaments, actin and microtubule cytoskeleton networks. It is required for anchoring either intermediate filaments to the actin cytoskeleton in neural and muscle cells or keratin-containing intermediate filaments to hemidesmosomes in epithelial cells.

### Epidermolysis Bullosa Simplex, Autosomal Recessive 2

In some embodiments, the invention encompasses methods to treat or alleviate conditions or complications associated with epidermolysis bullosa simplex, autosomal recessive 2. Epidermolysis bullosa simplex, autosomal recessive 2 (EBSB2) is a form of epidermolysis bullosa, a dermatologic disorder characterized by localized blistering on the dorsal, lateral and plantar surfaces of the feet. Epidermolysis bullosa simplex, autosomal recessive 2 is characterized by trauma-induced blistering mainly occurring on the feet and ankles. In subjects with epidermolysis bullosa simplex, autosomal recessive 2, ultrastructural analysis of skin biopsy shows abnormal hemidesmosomes with poorly formed inner plaques. Epidermolysis bullosa simplex, autosomal recessive 2 is associated with mutations of the BPAG1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolysis bullosa simplex, autosomal recessive 2, or who has been diagnosed with epidermolysis bullosa simplex, autosomal recessive 2.

In some embodiments, microvesicles are administered to a subject in need thereof to alleviate the symptoms or complications associated with epidermolysis bullosa simplex, autosomal recessive 2. In some embodiments the symptoms associated with epidermolysis bullosa simplex, autosomal recessive 2 include blistering on the dorsal, lateral and plantar surfaces of the feet, trauma-induced blistering on the feet and ankles, and abnormal hemidesmosomes with poorly formed inner plaques.

### Neuropathy, Hereditary Sensory and Autonomic, 6

In some embodiments, the invention encompasses methods to treat or alleviate conditions or complications associated with neuropathy, hereditary sensory and autonomic, 6. Neuropathy, hereditary sensory and autonomic, 6 (HSAN6) is a form of hereditary sensory and autonomic neuropathy, which is a genetically and clinically heterogeneous group of disorders characterized by degeneration of dorsal root and autonomic ganglion cells, and by sensory and/or autonomic abnormalities. Neuropathy, hereditary sensory and autonomic, 6 is a severe autosomal recessive disorder characterized by neonatal hypotonia, respiratory and feeding difficulties, lack of psychomotor development, autonomic abnormalities including labile cardiovascular function, lack of corneal reflexes leading to corneal scarring, areflexia, and absent axonal flare response after intradermal histamine injection. Neuropathy, hereditary sensory and autonomic, 6 is associated with mutations of the BPAG1 gene.

The methods featured in the invention include administering to a subject in need thereof a therapeutic composition comprising microvesicles. In some embodiments “a subject in need thereof” means a human or non-human animal that exhibits one or more symptoms or indicia of neuropathy, hereditary sensory and autonomic, 6, or who has been diagnosed with neuropathy, hereditary sensory and autonomic, 6.

In some embodiments, microvesicles are administered to a subject in need thereof to alleviate the symptoms or complications associated with neuropathy, hereditary sensory and autonomic, 6. In some embodiments the symptoms associated with neuropathy, hereditary sensory and autonomic, 6 are selected from the group consisting of degeneration of dorsal root and autonomic ganglion cells, sensory abnormalities, and autonomic abnormalities.

### Methods for Treating Keratin 1 Related Conditions

In some embodiments, the invention provides methods for treating epidermolytic hyperkeratosis, wherein the methods comprise administering a therapeutically effective amount of microvesicles to the subject.

In some embodiments, the subject has a mutation in the KRT1 gene.

In some embodiments, the microvesicles deliver keratin 1 protein to the cells of the subject. Keratins are a group of fibrous proteins that form structural frameworks for keratinocytes to make up the skin, hair, and nails. Keratin 1 partners with either keratin 9 or 10 to form heterodimer intermediate filaments, which then assemble into strong networks that provide tensile strength and resiliency to the skin to protect it from external damage.

### Epidermolytic Hyperkeratosis

In some embodiments, the invention encompasses methods to treat or alleviate conditions or complications associated with epidermolytic hyperkeratosis. Defects in keratin 1 are a cause of epidermolytic hyperkeratosis, also known as bullous congenital ichthyosiform erythroderma. Epidermolytic hyperkeratosis is a hereditary skin disorder characterized by intraepidermal blistering, a marked thickening of the stratum corneum, pigmentation of the skin, and erosions at sites of trauma which are all present from birth. Epidermolytic hyperkeratosis is associated with mutations of the KRT1 gene.

The methods featured in the invention include administering to a subject in need thereof a therapeutic composition comprising microvesicles. In some embodiments “a subject in need thereof” means a human or non-human animal that exhibits one or more symptoms or indicia of epidermolytic hyperkeratosis, or who has been diagnosed with epidermolytic hyperkeratosis.

In some embodiments, microvesicles are administered to a subject in need thereof to alleviate the symptoms or complications associated with epidermolytic hyperkeratosis. In some embodiments the symptoms associated with epidermolytic hyperkeratosis include intraepidermal blistering, thickening of the stratum corneum, pigmentation of the skin and erosions at sites of trauma, and erythroderma.

### Methods for Treating hSPCA1 Related Conditions

In some embodiments, the invention provides methods for treating benign familial pemphigus, wherein the methods comprise administering a therapeutically effective amount of microvesicles to the subject.

In some embodiments, the subject has a mutation in the ATP2C1 gene.

In some embodiments, the microvesicles deliver hSPCA1 protein to the cells of the subject. HSPCA1 protein is also known as calcium-transporting ATPase and is a magnesium-dependent enzyme that catalyzes the hydrolysis of ATP coupled with the transport of calcium.

### Benign Familial Pemphigus

In some embodiments, the invention encompasses methods to treat or alleviate conditions or complications associated with benign familial pemphigus. Benign familial pemphigus, also known as, Hailey-Hailey disease is a rare skin condition that usually appears in early adulthood. The disorder is characterized by red, raw, and blistered areas of skin that occur most often in skin folds. Benign familial pemphigus is associated with mutations of the ATP2C1 gene.

The methods featured in the invention include administering to a subject in need thereof a therapeutic composition comprising microvesicles. In some embodiments “a subject in need thereof” means a human or non-human animal that exhibits one or more symptoms or indicia of benign familial pemphigus, or who has been diagnosed with benign familial pemphigus.

In some embodiments, microvesicles are administered to a subject in need thereof to alleviate the symptoms or complications associated with benign familial pemphigus. In some embodiments the symptoms associated with benign familial pemphigus include blisters, erosions of the skin, rash, cracked skin, and acantholysis.

### Methods for Treating Lysosomal Trafficking Regulator Related Conditions

In some embodiments, the invention provides methods for treating Chediak-Higashi syndrome, wherein the methods comprise administering a therapeutically effective amount of microvesicles to the subject.

In some embodiments, the subject has a mutation in the LYST gene, also known as CHS.

In some embodiments, the microvesicles deliver lysosomal trafficking regulator protein to the cells of the subject. Lysosomal trafficking regulator may be required for sorting endosomal resident proteins into late multivesicular endosomes by a mechanism involving microtubules.

### Chediak-Higashi Syndrome

In some embodiments, the invention encompasses methods to treat or alleviate conditions or complications associated with Chediak-Higashi syndrome. Chediak-Higashi syndrome is a rare autosomal recessive disorder. Most patients die at an early age unless they receive an allogeneic hematopoietic stem cell transplant. Chediak-Higashi syndrome is associated with mutations of the LYST gene.

The methods featured in the invention include administering to a subject in need thereof a therapeutic composition comprising microvesicles. In some embodiments “a subject in need thereof” means a human or non-human animal that exhibits one or more symptoms or indicia of Chediak-Higashi syndrome, or who has been diagnosed with Chediak-Higashi syndrome.

In some embodiments, microvesicles are administered to a subject in need thereof to alleviate the symptoms or complications associated with Chediak-Higashi syndrome. In some embodiments the symptoms associated with Chediak-Higashi syndrome include hypopigmentation, severe immunologic deficiency, bleeding tendency, neurologic abnormalities, abnormal intracellular transport to and from the lysosome, and giant inclusion bodies in a variety of cell types.

### Methods for Treating Serine-Protein Kinase ATM Related Conditions

In some embodiments, the invention provides methods for treating ataxia telangiectasia syndrome; T-cell acute lymphoblastic leukemia; T-cell prolymphocytic leukemia; and B-cell chronic lymphocytic leukemia, wherein the methods comprise administering a therapeutically effective amount of microvesicles to the subject.

In some embodiments, the subject has a mutation in the ATM gene.

In some embodiments, the microvesicles deliver serine-protein kinase ATM (Ataxia telangiectasia mutated) protein to the cells of the subject. Serine-protein kinase ATM is a serine/threonine protein kinase which activates checkpoint signaling upon double strand breaks (DSBs), apoptosis, and genotoxic stresses such as ionizing ultraviolet A light (UVA), thereby acting as a DNA damage sensor.

### Ataxia Telangiectasia Syndrome

In some embodiments, the invention encompasses methods to treat or alleviate conditions or complications associated with ataxia telangiectasia syndrome. Ataxia telangiectasia (AT) is a rare recessive disorder. Patients have a strong predisposition to cancer, and about 30% of patients develop tumors, particularly lymphomas and leukemias. Cells from affected individuals are highly sensitive to damage by ionizing radiation and resistant to inhibition of DNA synthesis following irradiation.

Ataxia telangiectasia syndrome is associated with mutations of the ATM gene.

The methods featured in the invention include administering to a subject in need thereof a therapeutic composition comprising microvesicles. In some embodiments “a subject in need thereof” means a human or non-human animal that exhibits one or more symptoms or indicia of ataxia telangiectasia syndrome, or who has been diagnosed with ataxia telangiectasia syndrome.

In some embodiments, microvesicles are administered to a subject in need thereof to alleviate the symptoms or complications associated with ataxia telangiectasia syndrome. In some embodiments the symptoms associated with ataxia telangiectasia syndrome include progressive cerebellar ataxia, dilation of the blood vessels in the conjunctiva and eyeballs, immunodeficiency, growth retardation, and sexual immaturity.

### T-Cell Acute Lymphoblastic Leukemia

In some embodiments, the invention encompasses methods to treat or alleviate conditions or complications associated with T-cell acute lymphoblastic leukemia. T-cell acute lymphoblastic leukemia (T-ALL) is a type of acute leukemia meaning that it is aggressive and progresses quickly. It affects the lymphoid-cell-producing stem cells, in particular a type of white blood cell called T lymphocytes. T-cell acute lymphoblastic leukemia is associated with mutations of the ATM gene.

The methods featured in the invention include administering to a subject in need thereof a therapeutic composition comprising microvesicles. In some embodiments “a subject in need thereof” means a human or non-human animal that exhibits one or more symptoms or indicia of T-cell acute lymphoblastic leukemia, or who has been diagnosed with T-cell acute lymphoblastic leukemia.

In some embodiments, microvesicles are administered to a subject in need thereof to alleviate the symptoms or complications associated with T-cell acute lymphoblastic leukemia. In some embodiments the symptoms associated with T-cell acute lymphoblastic leukemia include anemia, frequent infections due to the lack of normal white blood cells, frequent infections, fever, purpura, and nosebleeds and bleeding gums due to lack of platelets.

### T-Cell Prolymphocytic Leukemia

In some embodiments, the invention encompasses methods to treat or alleviate conditions or complications associated with T-cell prolymphocytic leukemia. The clinical course of T-cell prolymphocytic leukemia (TPLL) is highly aggressive, with poor response to chemotherapy and short survival time. T-cell prolymphocytic leukemia occurs both in adults as a sporadic disease and in younger ataxia telangiectasia patients.

T-cell prolymphocytic leukemia is associated with mutations of the ATM gene.

The methods featured in the invention include administering to a subject in need thereof a therapeutic composition comprising microvesicles. In some embodiments “a subject in need thereof” means a human or non-human animal that exhibits one or more symptoms or indicia of T-cell prolymphocytic leukemia, or who has been diagnosed with T-cell prolymphocytic leukemia.

In some embodiments, microvesicles are administered to a subject in need thereof to alleviate the symptoms or complications associated with T-cell prolymphocytic leukemia. In some embodiments the symptoms associated with T-cell prolymphocytic leukemia include high white blood cell count, a predominance of prolymphocytes, marked splenomegaly, lymphadenopathy, skin lesions, and serous effusion.

### B-Cell Chronic Lymphocytic Leukemia

In some embodiments, the invention encompasses methods to treat or alleviate conditions or complications associated with B-cell chronic lymphocytic leukemia. B-cell chronic lymphocytic leukemia (B-CLL) is a type of B-cell non-Hodgkin lymphoma and is characterized by a highly variable clinical presentation. B-cell chronic lymphocytic leukemia is the most common form of leukemia in the elderly. B-cell chronic lymphocytic leukemia is associated with mutations of the ATM gene.

The methods featured in the invention include administering to a subject in need thereof a therapeutic composition comprising microvesicles. In some embodiments “a subject in need thereof” means a human or non-human animal that exhibits one or more symptoms or indicia of B-cell chronic lymphocytic leukemia, or who has been diagnosed with B-cell chronic lymphocytic leukemia.

In some embodiments, microvesicles are administered to a subject in need thereof to alleviate the symptoms or complications associated with B-cell chronic lymphocytic leukemia. In some embodiments the symptoms associated with B-cell chronic lymphocytic leukemia include accumulation of mature CD5+B-lymphocytes, lymphadenopathy, immunodeficiency, and bone marrow failure.

### Methods for Treating Tuberin Related Conditions

In some embodiments, the invention provides methods for treating tuberous sclerosis 2, wherein the methods comprise administering a therapeutically effective amount of microvesicles to the subject.

In some embodiments, the subject has a mutation in the TSC2 gene.

In some embodiments, the microvesicles deliver tuberin protein to the cells of the subject. In complex with TSC1, tuberin inhibits the nutrient-mediated or growth factor-stimulated phosphorylation of S6K1 and EIF4EBP1 by negatively regulating mTORC1 signaling. Tuberin acts as a GTPase-activating protein (GAP) for the small GTPase RHEB, which is a direct activator of the protein kinase activity of mTORC1. Tuberin also stimulates the intrinsic GTPase activity of the Ras-related proteins RAP1A and RAB5.

### Tuberous Sclerosis 2

In some embodiments, the invention encompasses methods to treat or alleviate conditions or complications associated with tuberous sclerosis 2. Tuberous sclerosis 2 (TSC2) is an autosomal dominant multi-system disorder that especially effects the brain, kidneys, heart, and skin. Clinical manifestations include epilepsy, learning difficulties, behavioral problems, and skin lesions. Seizures can be intractable and premature death can occur from a variety of disease-associated causes. Tuberous sclerosis 2 is associated with mutations of the TSC2 gene.

The methods featured in the invention include administering to a subject in need thereof a therapeutic composition comprising microvesicles. In some embodiments “a subject in need thereof” means a human or non-human animal that exhibits one or more symptoms or indicia of tuberous sclerosis 2, or who has been diagnosed with tuberous sclerosis 2.

In some embodiments, microvesicles are administered to a subject in need thereof to alleviate the symptoms or complications associated with tuberous sclerosis 2. In some embodiments the symptoms associated with tuberous sclerosis 2 include hamartomas, hamartias, epilepsy, learning difficulties, behavioral problems, and skin lesions.

### Methods for Treating FOXM1A Related Conditions

In some embodiments, the invention provides methods for treating diabetic foot ulcers, wherein the methods comprise administering a therapeutically effective amount of microvesicles to the subject.

In some embodiments, the subject has a mutation in the FOXM1A gene.

In some embodiments, the microvesicles deliver FOXM1A protein to the cells of the subject. The transcription factor Forkhead box M1 (FOXM1) plays important roles in oncogenesis, FOXM1A is one of the FOXM1 isoforms.

### Diabetic Foot Ulcers

In some embodiments, the invention encompasses methods to treat or alleviate conditions or complications associated with diabetic foot ulcers. Foot ulcers are a common complication of poorly controlled diabetes, forming as a result of skin tissue breaking down and exposing the layers underneath. Type 2 diabetes incidence increases with age, while β-cell replication declines. Furthermore, the transcription factor FoxM1 is required for β-cell replication in various situations, and its expression declines with age. Therefore, an increase in FOXM1A protein may have a role in alleviating the symptoms associated with diabetic foot ulcers.

The methods featured in the invention include administering to a subject in need thereof a therapeutic composition comprising microvesicles. In some embodiments “a subject in need thereof” means a human or non-human animal that exhibits one or more symptoms or indicia of diabetic foot ulcers, or who has been diagnosed with diabetic foot ulcers.

In some embodiments, microvesicles are administered to a subject in need thereof to alleviate the symptoms or complications associated with diabetic foot ulcers. In some embodiments the symptoms associated with diabetic foot ulcers include open sores or wounds on the foot of the subject.

### Methods to Isolate the Microvesicles Described Herein

As used herein, the term “microvesicles” refers to vesicles comprising lipid bilayers, formed from the plasma membrane of cells. In some embodiments, microvesicles are heterogeneous in size, ranging from about 2 nm to about 5000 nm. The cell from which a microvesicle is formed is herein referred to as “the host cell.” Microvesicles include, but are not limited to, extracellular vesicles (EVs), ectosomes, microparticles, microvesicles, nanovesicles, shedding vesicles, membrane particles and the like.

Microvesicles exhibit membrane proteins from their host cell on their membrane surface, and may also contain molecules within the microvesicle from the host cell, such as, for example, mRNA, miRNA, tRNA, RNA, DNA, lipids, proteins or infectious particles. These molecules may result from, or be, recombinant molecules introduced into the host cell. Microvesicles play a critical role in intercellular communication, and can act locally and distally within the body, inducing changes in cells by fusing with a target cell, introducing the molecules transported on and/or in the microvesicle to the target cell. For example, microvesicles have been implicated in anti-tumor reversal, cancer, tumor immune suppression, metastasis, tumor-stroma interactions, angiogenesis and tissue regeneration. Microvesicles may also be used to diagnose disease, as they have been shown to carry bio-markers of several diseases, including, for example, cardiac disease, HIV and leukemia.

In some embodiments, the microvesicles are isolated according to the methods of U.S. Pat. No. 10,500,231, incorporated by reference herein in its entirety.

In one embodiment, microvesicles are isolated from a biological fluid containing microvesicles in a method comprising the steps of:

a) obtaining a biological fluid containing microvesicles,

b) clarifying the biological fluid to remove cellular debris,

c) precipitating the microvesicles by adding a precipitating agent to the clarified biological fluid,

d) collecting the precipitated microvesicles and washing the material to remove the precipitating agent, and

e) suspending the washed microvesicles in a solution for storage or subsequent use.

In one embodiment, the biological fluid is clarified by centrifugation. In an alternate embodiment, the biological fluid is clarified by filtration.

In one embodiment, the precipitated microvesicles are collected by centrifugation. In an alternate embodiment, the precipitated microvesicles are collected by filtration.

In one embodiment, microvesicles are isolated from a biological fluid containing microvesicles in a method comprising the steps of:

a) obtaining a biological fluid containing microvesicles,

b) clarifying the biological fluid to remove cellular debris,

c) precipitating the microvesicles by adding a precipitating agent to the clarified biological fluid,

d) collecting the precipitated microvesicles and washing the material to remove the precipitating agent,

e) suspending the washed microvesicles in a solution, and

f) processing the microvesicles to analyze the nucleic acid, carbohydrate, lipid, small molecules and/or protein content.

In one embodiment, the biological fluid is clarified by centrifugation. In an alternate embodiment, the biological fluid is clarified by filtration.

In one embodiment, the precipitated microvesicles are collected by centrifugation. In an alternate embodiment, the precipitated microvesicles are collected by filtration.

In one embodiment, the present disclosure provides reagents and kits to isolate microvesicles from biological fluids according to the methods described herein.

The biological fluid may be peripheral blood, sera, plasma, ascites, urine, cerebrospinal fluid (CSF), sputum, saliva, bone marrow, synovial fluid, aqueous humor, amniotic fluid, cerumen, breast milk, broncheo alveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids.

The biological fluid may also be derived from the blastocyl cavity, umbilical cord blood, or maternal circulation, which may be of fetal or maternal origin. The biological fluid may also be derived from a tissue sample or biopsy.

In some embodiments, the biological fluid is obtained from bone marrow or bone marrow aspirates. In one embodiment, the biological fluid is cell culture medium. In one embodiment, the cell culture medium is conditioned using tissues and/or cells prior to the isolation of microvesicles according to the methods described herein. In some embodiments, BM-MSCs obtained from bone marrow or bone marrow aspirate are cultured in culture media to allow for production and collection of the BM-MSC secretome. In some embodiments, the culture media is serum-free.

The term “conditioned” or “conditioned medium” refers to medium, wherein a population of cells or tissue, or combination thereof is grown, and the population of cells or tissue, or combination thereof contributes factors to the medium. In one such use, the population of cells or tissue, or combination thereof is removed from the medium, while the factors the cells produce remain. In one embodiment, the factors produced are microvesicles. Medium may be conditioned via any suitable method selected by one of ordinary skill in the art. For example, medium may be cultured according to the methods described in EP1780267A2, incorporated by reference herein in its entirety.

In one embodiment, microvesicles are isolated from cells or tissue that have been pre-treated prior to the isolation of the microvesicles. Pretreatment may include, for example, culture in a specific medium, a medium that contains at least one additive, growth factor, medium devoid of serum, or a combination thereof. Alternatively, pretreatment may comprise contacting cells or tissues with additives (e.g. interleukin, VEGF, inducers of transcription factors, transcription factors, hormones, neurotransmitters, pharmaceutical compounds, microRNA), transforming agents (e.g. liposome, viruses, transfected agents, etc.). Alternatively, pretreatment may comprise exposing cells or tissue to altered physical conditions (e.g. hypoxia, cold shock, heat shock and the like).

In one embodiment, microvesicles are isolated from medium conditioned using cells or tissue that have been pre-treated prior to the isolation of the microvesicles. Pretreatment may include, for example, culture in a specific medium, a medium that contains at least one additive, growth factor, medium devoid of serum, or a combination thereof. Alternatively, pretreatment may comprise contacting cells or tissues with additives (e.g. interleukin, VEGF, inducers of transcription factors, transcription factors, hormones, neurotransmitters, pharmaceutical compounds, microRNA), transforming agents (e.g. liposome, viruses, transfected agents, etc.). Alternatively, pretreatment may comprise exposing cells or tissue to altered physical conditions (e.g. hypoxia, cold shock, heat shock and the like).

While the methods described herein may be carried out at any temperature, one of ordinary skill in the art can readily appreciate that certain biological fluids may degrade, and such degradation is reduced if the sample is maintained at a temperature below the temperature at which the biological fluid degrades. In one embodiment, the method described herein is carried out at 4° C. In an alternate embodiment, at least one step of the method described herein is carried out at 4° C. In certain embodiments, the biological fluid may be diluted prior to being subjected to the methods described herein. Dilution may be required for viscous biological fluids, to reduce the viscosity of the sample, if the viscosity of the sample is too great to obtain an acceptable yield of microvesicles. The dilution may be a 1:2 dilution. Alternatively, the dilution may be a 1:3 dilution. Alternatively, the dilution may be a 1:4 dilution. Alternatively, the dilution may be a 1:5 dilution. Alternatively, the dilution may be a 1:6 dilution. Alternatively, the dilution may be a 1:7 dilution. Alternatively, the dilution may be a 1:8 dilution. Alternatively, the dilution may be a 1:9 dilution. Alternatively, the dilution may be a 1:10 dilution. Alternatively, the dilution may be a 1:20 dilution. Alternatively, the dilution may be a 1:30 dilution. Alternatively, the dilution may be a 1:40 dilution. Alternatively, the dilution may be a 1:50 dilution. Alternatively, the dilution may be a 1:60 dilution. Alternatively, the dilution may be a 1:70 dilution. Alternatively, the dilution may be a 1:80 dilution. Alternatively, the dilution may be a 1:90 dilution. Alternatively, the dilution may be a 1:100 dilution.

The biological fluid may be diluted with any diluent, provided the diluent does not affect the functional and/or structural integrity of the microvesicles. One of ordinary skill in the art may readily select a suitable diluent. Diluents may be, for example, phosphate buffered saline, cell culture medium, and the like.

In one embodiment, the biological fluid is clarified by the application of a centrifugal force to remove cellular debris. The centrifugal force applied to the biological fluid is sufficient to remove any cells, lysed cells, tissue debris from the biological fluid, but the centrifugal force applied is insufficient in magnitude, duration, or both, to remove the microvesicles. The biological fluid may require dilution to facilitate the clarification.

The duration and magnitude of the centrifugal force used to clarify the biological fluid may vary according to a number of factors readily appreciated by one of ordinary skill in the art, including, for example, the biological fluid, the pH of the biological fluid, the desired purity of the isolated microvesicles, the desired size of the isolated microvesicles, the desired molecular weight of the microvesicles, and the like. In one embodiment, a centrifugal force of 2000×g is applied to the biological fluid for 30 minutes.

The clarified biological fluid is contacted with a precipitation agent to precipitate the microvesicles. In one embodiment, the precipitation agent may be any agent that surrounds the microvesicles and displaces the water of solvation. Such precipitation agents may be selected from the group consisting of polyethylene glycol, dextran, and polysaccharides.

In an alternate embodiment, the precipitation agent may cause aggregation of the microvesicles.

In an alternate embodiment, the precipitation agent is selected from the group consisting of calcium ions, magnesium ions, sodium ions, ammonium ions, iron ions, organic solvents such as ammonium sulfate, and flocculating agents, such as alginate.

The clarified biological fluid is contacted with the precipitation agent for a period of time sufficient to precipitate the microvesicles. The period of time sufficient to precipitate the microvesicles may vary according to a number of factors readily appreciated by one of ordinary skill in the art, including, for example, the biological fluid, the pH of the biological fluid, the desired purity of the isolated microvesicles, the desired size of the isolated microvesicles, the desired molecular weight of the microvesicles, and the like. In one embodiment, the period of time sufficient to precipitate the microvesicles is 6 hours.

In one embodiment, the clarified biological fluid is contacted with the precipitation agent for a period of time sufficient to precipitate the microvesicles at 4° C.

The concentration of the precipitation agent used to precipitate the microvesicles from a biological fluid may vary according to a number of factors readily appreciated by one of ordinary skill in the art, including, for example, the biological fluid, the pH of the biological fluid, the desired purity of the isolated microvesicles, the desired size of the isolated microvesicles, the desired molecular weight of the microvesicles, and the like.

In one embodiment, the precipitation agent is polyethylene glycol. The molecular weight of polyethylene glycol used in the methods described herein may be from about 200 Da to about 10,000 Da. In one embodiment, the molecular weight of polyethylene glycol used in the methods described herein may be greater than 10,000 Da. In certain embodiments, the molecular weight of polyethylene glycol used in the methods described herein is 10,000 Da or 20,000 Da. The choice of molecular weight may be influenced by a variety of factors including, for example, the viscosity of the biological fluid, the desired purity of the microvesicles, the desired size of the microvesicles, the biological fluid used, and the like. In one embodiment, the molecular weight of polyethylene glycol used in the methods described herein may be from about 200 Da to about 8,000 Da, or is approximately any of 200 Da, 300 Da, 400 Da, 600 Da, 1000 Da, 1450 Da, 1500 Da, 2000 Da, 3000 Da, 3350 Da, 4000 Da, 6000 Da, 8000 Da, 10000 Da, 20000 Da or 35000 Da or any ranges or molecular weights in between.

In one embodiment, the molecular weight of polyethylene glycol used in the methods described herein is about 6000 Da.

In one embodiment, the average molecular weight of polyethylene glycol used in the methods described herein is about 8000 Da.

In one embodiment, the average molecular weight of polyethylene glycol used in the methods described herein is about 10000 Da.

In one embodiment, the average molecular weight of polyethylene glycol used in the methods described herein is about 20000 Da.

The concentration of polyethylene glycol used in the methods described herein may be from about 0.5% w/v to about 100% w/v. The concentration of polyethylene glycol used in the methods described herein may be influenced by a variety of factors including, for example, the viscosity of the biological fluid, the desired purity of the microvesicles, the desired size of the microvesicles, the biological fluid used, and the like.

In certain embodiments, the polyethylene glycol is used in the concentration described herein at a concentration between about 5% and 25% w/v. In certain embodiments, the concentration is about 5%, 6%, 7%, 8%, 9%, 10%, 11%, 12%, 13%, 14%, or 15%, or a range between any two of these values.

In one embodiment, the concentration of polyethylene glycol used in the methods described herein is about 8.5% w/v.

In one embodiment, the concentration of polyethylene glycol used in the methods described herein is about 6% w/v.

In one embodiment, polyethylene glycol having an average molecular weight of 6000 Da is used, at a concentration of 8.5% w/v. In one embodiment, the polyethylene glycol is diluted in 0.4M sodium chloride.

In one embodiment, the concentration of the polyethylene glycol used in the methods described herein is inversely proportional to the average molecular weight of the polyethylene glycol. For example, in one embodiment, polyethylene glycol having an average molecular weight of 4000 Da is used, at a concentration of 20% w/v. In an alternate embodiment, polyethylene glycol having an average molecular weight of 8000 Da is used, at a concentration of 10% w/v. In an alternate embodiment, polyethylene glycol having an average molecular weight of 20000 Da is used, at a concentration of 4% w/v.

In one embodiment, the precipitated microvesicles are collected by the application of centrifugal force. The centrifugal force is sufficient and applied for a duration sufficient to cause the microvesicles to form a pellet, but insufficient to damage the microvesicles.

The duration and magnitude of the centrifugal force used to precipitate the microvesicles from a biological fluid may vary according to a number of factors readily appreciated by one of ordinary skill in the art, including, for example, the biological fluid, the pH of the biological fluid, the desired purity of the isolated microvesicles, the desired size of the isolated microvesicles, the desired molecular weight of the microvesicles, and the like. In one embodiment, the precipitated microvesicles are collected by the application of a centrifugal force of 10000×g for 60 minutes.

The precipitated microvesicles may be washed with any liquid, provided the liquid does not affect the functional and/or structural integrity of the microvesicles. One of ordinary skill in the art may readily select a suitable liquid. Liquids may be, for example, phosphate buffered saline, cell culture medium, and the like.

In one embodiment, the washing step removes the precipitating agent. In one embodiment, the microvesicles are washed via centrifugal filtration, using a filtration device with a 100 kDa molecular weight cut off.

The isolated microvesicles may be suspended with any liquid, provided the liquid does not affect the functional and/or structural integrity of the microvesicles. One of ordinary skill in the art may readily select a suitable liquid. Liquids may be, for example, phosphate buffered saline, cell culture medium, and the like.

In one embodiment, the isolated microvesicles may be further processed. The further processing may be the isolation of a microvesicle of a specific size. Alternatively, the further processing may be the isolation of microvesicles of a particular size range. Alternatively, the further processing may be the isolation of a microvesicle of a particular molecular weight. Alternatively, the further processing may be the isolation of microvesicles of a particular molecular weight range. Alternatively, the further processing may be the isolation of a microvesicle exhibiting or containing a specific molecule.

In one embodiment, the microvesicles described herein are further processed to isolate a preparation of microvesicles having a size of about 2 nm to about 1000 nm as determined by electron microscopy. In an alternate embodiment, the microvesicles described herein are further processed to isolate a preparation of microvesicles having a size of about 2 nm to about 500 nm as determined by electron microscopy. In an alternate embodiment, the microvesicles described herein are further processed to isolate a preparation of microvesicles having a size of about 2 nm to about 400 nm as determined by electron microscopy. In an alternate embodiment, the microvesicles described herein are further processed to isolate a preparation of microvesicles having a size of about 2 nm to about 300 nm as determined by electron microscopy. In an alternate embodiment, the microvesicles described herein are further processed to isolate a preparation of microvesicles having a size of about 2 nm to about 200 nm as determined by electron microscopy. In an alternate embodiment, the microvesicles described herein are further processed to isolate a preparation of microvesicles having a size of about 2 nm to about 100 nm as determined by electron microscopy. In an alternate embodiment, the microvesicles described herein are further processed to isolate a preparation of microvesicles having a size of about 2 nm to about 50 nm as determined by electron microscopy. In an alternate embodiment, the microvesicles described herein are further processed to isolate a preparation of microvesicles having a size of about 2 nm to about 20 nm as determined by electron microscopy. In an alternate embodiment, the microvesicles described herein are further processed to isolate a preparation of microvesicles having a size of about 2 nm to about 10 nm as determined by electron microscopy.

In one embodiment, the subsequent purification is performed using a method selecting from the group consisting of immunoaffinity, HPLC, tangential flow filtration, phase separation/partitioning, and microfluidics.

In one embodiment, the isolated microvesicles are further processed to analyze the molecules exhibited on, or contained within the microvesicles. The molecules analyzed are selected from the group consisting of nucleic acid, carbohydrate, lipid, small molecules, ions, metabolites, protein, and combinations thereof.

In one embodiment, microvesicles are obtained from medium conditioned using cultured cells. Any cultured cell, or population of cells may be used in the methods described herein. The cells may be stem cells, primary cells, cell lines, tissue or organ explants, or any combination thereof. The cells may be allogeneic, autologous, or xenogeneic in origin.

In one embodiment, the cells are cells derived from bone-marrow aspirate. In one embodiment, the cells derived from bone marrow aspirate are bone marrow-derived mesenchymal stem cells. In one embodiment, the cells derived from bone marrow aspirate are mononuclear cells. In one embodiment, the cells derived from bone marrow aspirate are a mixture of mononuclear cells and bone marrow-derived mesenchymal stem cells.

In one embodiment, bone marrow-derived mesenchymal stem cells are isolated from bone marrow aspirate by culturing bone marrow aspirate in plastic tissue culture flasks for a period of time of up to about 4 days, followed by a wash to remove the non-adherent cells.

In one embodiment, mononuclear cells are isolated from bone marrow aspirate by low-density centrifugation using a Ficoll gradient, and collecting the mononuclear cells at the interface.

In one embodiment, prior to isolation of microvesicles according to the methods described herein, the cells are cultured, grown or maintained at an appropriate temperature and gas mixture (typically, 37° C., 5% CO₂ for mammalian cells) in a cell incubator. Culture conditions vary widely for each cell type, and are readily determined by one of ordinary skill in the art.

In one embodiment, one, or more than one culture condition is varied. In one embodiment, this variation results in a different phenotype.

In one embodiment, where the cells require serum in their culture medium, to begin the microvesicle isolation procedure, the cell culture medium is supplemented with microvesicle-free serum and then added to the cells to be conditioned. The microvesicles are collected from the conditioned cell culture medium. Serum may be depleted by any suitable method, such as, for example, ultracentrifugation, filtration, precipitation, and the like. The choice of medium, serum concentration, and culture conditions are influenced by a variety of factors readily appreciated by one of ordinary skill in the art, including, for example, the cell type being cultured, the desired purity of the microvesicles, the desired phenotype of the cultured cell, and the like. In one embodiment, the cell culture medium that is conditioned for the microvesicle isolation procedure is the same type of cell culture medium that the cells were grown in, prior to the microvesicle isolation procedure.

In one embodiment, to begin the microvesicle isolation procedure, the cell culture medium is removed, and serum-free medium is added to the cells to be conditioned. The microvesicles are then collected from the conditioned serum free medium. The choice of medium, and culture conditions are influenced by a variety of factors readily appreciated by one of ordinary skill in the art, including, for example, the cell type being cultured, the desired purity of the microvesicles, the desired phenotype of the cultured cell, and the like. In one embodiment, the serum-free medium is supplemented with at least one additional factor that promotes or enhances the survival of the cells in the serum free medium. Such factor may, for example, provide trophic support to the cells, inhibit, or prevent apoptosis of the cells.

The cells are cultured in the culture medium for a period of time sufficient to allow the cells to secrete microvesicles into the culture medium. The period of time sufficient to allow the cells to secrete microvesicles into the culture medium is influenced by a variety of factors readily appreciated by one of ordinary skill in the art, including, for example, the cell type being cultured, the desired purity of the microvesicles, the desired phenotype of the cultured cell, desired yield of microvesicles, and the like.

The microvesicles are then removed from the culture medium by the methods described herein.

In one embodiment, prior to the microvesicle isolation procedure, the cells are treated with at least one agent selected from the group consisting of an anti-inflammatory compound, an anti-apoptotic compound, an inhibitor of fibrosis, a compound that is capable of enhancing angiogenesis, an immunosuppressive compound, a compound that promotes survival of the cells, a chemotherapeutic, a compound capable of enhancing cellular migration, a neurogenic compound, and a growth factor. In one embodiment, while the cells are being cultured in the medium from which the microvesicles are collected, the cells are treated with at least one agent selected from the group consisting of an anti-inflammatory compound, an anti-apoptotic compound, an inhibitor of fibrosis, a compound that is capable of enhancing angiogenesis, an immunosuppressive compound, a compound that promotes survival of the cells, and a growth factor.

In one embodiment, the anti-inflammatory compound may be selected from the compounds disclosed in U.S. Pat. No. 6,509,369, incorporated by reference herein in its entirety.

In one embodiment, the anti-apoptotic compound may be selected from the compounds disclosed in U.S. Pat. No. 6,793,945, incorporated by reference herein in its entirety.

In one embodiment, the inhibitor of fibrosis may be selected from the compounds disclosed in U.S. Pat. No. 6,331,298, incorporated by reference herein in its entirety.

In one embodiment, the compound that is capable of enhancing angiogenesis may be selected from the compounds disclosed in U. S. Patent Application 2004/0220393 or U. S. Patent Application 2004/0209901, incorporated by reference herein in their entireties.

In one embodiment, the immunosuppressive compound may be selected from the compounds disclosed in U. S. Patent Application 2004/0171623, incorporated by reference herein in its entirety.

In one embodiment, the compound that promotes survival of the cells may be selected from the compounds disclosed in U. S. Patent Application 2010/0104542, incorporated by reference herein in its entirety.

In one embodiment, the growth factor may be at least one molecule selected from the group consisting of members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, -AB, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, hypoxia inducible factor 1-alpha, glucagon like peptide-1 (GLP-1), GLP-1 and GLP-2 mimetibody, and II, Exendin-4, nodal, noggin, NGF, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, and MAPK inhibitors, such as, for example, compounds disclosed in U. S. Patent Application 2004/0209901 and U. S. Patent Application 2004/0132729, incorporated by reference herein in their entireties.

In one embodiment, microvesicles are isolated from a biological fluid comprising cell culture medium conditioned using a culture of bone marrow-derived mesenchymal stem cells comprising the steps of:

a) obtaining a population of bone marrow-derived mesenchymal stem cells and seeding flasks at a 1:4 dilution of cells,

b) culturing the cells in medium until the cells are 80 to 90% confluent,

c) removing and clarifying the medium to remove cellular debris,

d) precipitating the microvesicles by adding a precipitating agent to the clarified culture medium,

e) collecting the precipitated microvesicles and washing the material to remove the precipitating agent, and

f) suspending the washed microvesicles in a solution for storage or subsequent use.

In one embodiment, microvesicles are isolated from a biological fluid comprising cell culture medium conditioned using a culture of bone marrow-derived mononuclear cells comprising the steps of:

a) obtaining a population of bone marrow-derived mononuclear cells and seeding flasks at a 1:4 dilution of cells,

b) culturing the cells in medium until the cells are 80 to 90% confluent,

c) removing and clarifying the medium to remove cellular debris,

d) precipitating the microvesicles by adding a precipitating agent to the clarified culture medium,

e) collecting the precipitated microvesicles and washing the material to remove the precipitating agent, and

f) suspending the washed microvesicles in a solution for storage or subsequent use.

In one embodiment, the bone marrow-derived mesenchymal stem cells are cultured in medium comprising α-MEM supplemented with 20% fetal bovine serum and 1% penicillin/streptomycin/glutamine at 37° C. in 95% humidified air and 5% CO₂.

In one embodiment, the bone marrow-derived mononuclear cells are cultured in medium comprising α-MEM supplemented with 20% fetal bovine serum and 1% penicillin/streptomycin/glutamine at 37° C. in 95% humidified air and 5% CO₂.

In one embodiment, the medium is clarified by centrifugation.

In one embodiment, the precipitating agent is polyethylene glycol having an average molecular weight of 6000. In one embodiment, the polyethylene glycol is used at a concentration of about 8.5 w/v %. In one embodiment, the polyethylene glycol is diluted in a sodium chloride solution having a final concentration of 0.4 M.

In one embodiment, the precipitated microvesicles are collected by centrifugation.

In one embodiment, the isolated microvesicles are washed via centrifugal filtration, using a membrane with a 100 kDa molecular weight cut-off, using phosphate buffered saline.

Biological fluid comprising plasma: In one embodiment, microvesicles are obtained from plasma. The plasma may be obtained from a healthy individual, or, alternatively, from an individual with a particular disease phenotype.

In one embodiment, microvesicles are isolated from a biological fluid comprising plasma comprising the steps of:

a) obtaining plasma and diluting the plasma with cell culture medium,

b) precipitating the microvesicles by adding a precipitating agent to the diluted plasma,

c) collecting the precipitated microvesicles and washing the material to remove the precipitating agent, and

d) suspending the washed microvesicles in a solution for storage or subsequent use.

In one embodiment, the plasma is diluted 1:10 with culture medium. In one embodiment, the culture medium is α-MEM.

In some embodiments, the microvesicles are isolated from plasma according to the methods of U.S. Pat. No. 10,500,231, incorporated by reference herein in its entirety.

In some embodiments, the microvesicles are isolated from urine according to the methods of U.S. Pat. No. 10,500,231, incorporated by reference herein in its entirety.

In one embodiment, the precipitating agent is polyethylene glycol having an average molecular weight of 6000. In one embodiment, the polyethylene glycol is used at a concentration of about 8.5 w/v %. In one embodiment, the polyethylene glycol is diluted in a sodium chloride solution having a final concentration of 0.4 M.

In one embodiment, the precipitated microvesicles are collected by centrifugation.

In one embodiment, the isolated microvesicles are washed via centrifugal filtration, using a membrane with a 100 kDa molecular weight cut-off, using phosphate buffered saline.

Biological fluid comprising bone marrow aspirate: In one embodiment, microvesicles are obtained from bone marrow aspirate. In one embodiment, microvesicles are obtained from the cellular fraction of the bone marrow aspirate. In one embodiment, microvesicles are obtained from the acellular fraction of the bone marrow aspirate.

In one embodiment, microvesicles are obtained from cells cultured from bone marrow aspirate. In one embodiment, the cells cultured from bone marrow aspirate are used to condition cell culture medium, from which the microvesicles are isolated.

In one embodiment, microvesicles are isolated from a biological fluid comprising bone marrow aspirate comprising the steps of:

a) obtaining bone marrow aspirate and separating the bone marrow aspirate into an acellular portion and a cellular portion,

b) diluting the acellular portion,

c) clarifying the diluted acellular portion to remove cellular debris,

d) precipitating the microvesicles in the acellular portion by adding a precipitating agent to the diluted acellular portion,

e) collecting the precipitated microvesicles and washing the material to remove the precipitating agent, and

f) suspending the washed microvesicles in a solution for storage or subsequent use.

In one embodiment, the acellular portion is diluted 1:10 with culture medium.

In one embodiment, the culture medium is α-MEM.

In one embodiment, the diluted acellular portion is clarified by centrifugation.

In one embodiment, the precipitating agent is polyethylene glycol having an average molecular weight of 6000. In one embodiment, the polyethylene glycol is used at a concentration of about 8.5 w/v %. In one embodiment, the polyethylene glycol is diluted in a sodium chloride solution having a final concentration of 0.4 M.

In one embodiment, the precipitated microvesicles are collected by centrifugation.

In one embodiment, the isolated microvesicles are washed via centrifugal filtration, using a membrane with a 100 kDa molecular weight cut-off, using phosphate buffered saline.

In one embodiment the cellular portion is further processed to isolate and collect cells. In one embodiment, the cellular portion is further processed to isolate and collect bone marrow-derived mesenchymal stem cells. In one embodiment, the cellular portion is further processed to isolate and collect bone marrow-derived mononuclear cells. In one embodiment, the cellular portion is used to condition medium, from which microvesicles may later be derived.

In one embodiment, microvesicles are isolated from the cellular portion. The cellular portion may be incubated for a period of time prior to the isolation of the microvesicles. Alternatively, the microvesicles may be isolated from the cellular portion immediately after the cellular portion is collected.

In some embodiments, the microvesicles are isolated from culture medium conditioned using bone marrow derived stem cells according to the methods of U.S. Pat. No. 10,500,231, incorporated by reference herein in its entirety. In some embodiments, the microvesicles are isolated from culture medium conditioned using bone marrow aspirate according to the methods of U.S. Pat. No. 10,500,231, incorporated by reference herein in its entirety. In some embodiments, the microvesicles are isolated from culture medium from a long-term culture of bone marrow cells according to the methods of U.S. Pat. No. 10,500,231, incorporated by reference herein in its entirety.

In one embodiment, the cellular portion is also treated with at least one agent selected from the group consisting of an anti-inflammatory compound, an anti-apoptotic compound, an inhibitor of fibrosis, a compound that is capable of enhancing angiogenesis, an immunosuppressive compound, a compound that promotes survival of the cells, a chemotherapeutic, a compound capable of enhancing cellular migration, a neurogenic compound, and a growth factor.

In one embodiment, the anti-inflammatory compound may be selected from the compounds disclosed in U.S. Pat. No. 6,509,369, incorporated by reference herein in its entirety.

In one embodiment, the anti-apoptotic compound may be selected from the compounds disclosed in U.S. Pat. No. 6,793,945, incorporated by reference herein in its entirety.

In one embodiment, the inhibitor of fibrosis may be selected from the compounds disclosed in U.S. Pat. No. 6,331,298, incorporated by reference herein in its entirety.

In one embodiment, the compound that is capable of enhancing angiogenesis may be selected from the compounds disclosed in U. S. Patent Application 2004/0220393 or U. S. Patent Application 2004/0209901, incorporated by reference herein in their entireties.

In one embodiment, the immunosuppressive compound may be selected from the compounds disclosed in U. S. Patent Application 2004/0171623, incorporated by reference herein in its entirety.

In one embodiment, the compound that promotes survival of the cells may be selected from the compounds disclosed in U. S. Patent Application 2010/0104542, incorporated by reference herein in its entirety.

In one embodiment, the growth factor may be at least one molecule selected from the group consisting of members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, -AB, and —BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, hypoxia inducible factor 1-alpha, glucagon like peptide-1 (GLP-1), GLP-1 and GLP-2 mimetibody, and II, Exendin-4, nodal, noggin, NGF, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, and MAPK inhibitors, such as, for example, compounds disclosed in U. S. Patent Application 2004/0209901 and U. S. Patent Application 2004/0132729, incorporated by reference herein in their entireties. In one embodiment, the cellular portion is cultured under hypoxic conditions. In one embodiment, the cellular portion is heat-shocked.

In some embodiments, the microvesicles are isolated from cell culture by ultracentrifugation. In some embodiments, the microvesicles are isolated from cell culture by ultracentrifugation according to the methods of U.S. Pat. No. 10,500,231, incorporated by reference herein in its entirety.

In one embodiment, the microvesciles are isolated from cell culture by ultracentrifugation according to the following method:

The cells are cultured in medium supplemented with microvesicle-free serum (the serum may be depleted of microvesicles by ultracentrifugation, filtration, precipitation, etc.). After culturing the cells for a period of time, the medium is removed and transferred to conical tubes and centrifuged at 400×g for 10 minutes at 4° C. to pellet the cells. Next, the supernatant is transferred to new conical tubes and centrifuged at 2000×g for 30 minutes at 4° C. to further remove cells and cell debris. This may be followed by another centrifugation step (e.g. 10000×g for 30 minutes to further deplete cellular debris and/or remove larger microvesicles). The resultant supernatant is transferred to ultracentrifuge tubes, weighed to ensure equal weight and ultracentrifuged at 70000+×g for 70 minutes at 4° C. to pellet the microvesicles. This supernatant is subsequently discarded and the pellet is resuspended in ice cold PBS. The solution is ultracentrifuged at 70000+×g for 70 minutes at 4° C. to pellet the microvesicles. The microvesicle enriched pellet is resuspended in a small volume (approximately 50-100 μl) of an appropriate buffer (e.g. PBS).

In one embodiment, the precipitating agent is polyethylene glycol having an average molecular weight of 6000. In one embodiment, the polyethylene glycol is used at a concentration of about 8.5 w/v %. In one embodiment, the polyethylene glycol is diluted in a sodium chloride solution having a final concentration of 0.4 M.

In some embodiments, the microvesciles are precipitated by polyethylene glycol according to the methods of U.S. Pat. No. 10,500,231, incorporated by reference herein in its entirety. In one embodiment, the microvesciles are precipitated by polyethylene glycol according to the following method:

The cells are cultured in medium supplemented with microvesicle-free serum (the serum may be depleted of microvesicles by ultracentrifugation, filtration, precipitation, etc.). After culturing the cells for a period of time, the medium is removed and transferred to conical tubes and centrifuged at 400×g for 10 minutes at 4° C. to pellet the cells. Next, the supernatant is transferred to new conical tubes and centrifuged at 2000×g for 30 minutes at 4° C. to further remove cells and cell debris. This may be followed by another centrifugation step (e.g. 10000×g for 30 minutes to further deplete cellular debris and remove larger particles).

Microvesicles are then precipitated at 4° C. using 8.5% w/v PEG 6000 and 0.4 M NaCl. This mixture is spun at 10000×g at 4° C. for 30 minutes. The supernatant is removed and the pellet is resuspended in an appropriate buffer (e.g. PBS). It may be used for immediate downstream reactions or further purified. Further purification procedures can include the use of centrifugal filters (e.g. MWCO of 100 kDa), immunoaffinity, HPLC, tangential flow filtration, phase separation/partitioning, microfluidics, etc.

In one embodiment, the precipitated microvesicles are collected by centrifugation.

In one embodiment, the isolated microvesicles are washed via centrifugal filtration, using a membrane with a 100 kDa molecular weight cut-off, using phosphate buffered saline.

In an alternate embodiment described herein, the biological fluids are clarified by filtration. In an alternate embodiment, the precipitated microvesicles are collected by filtration. In an alternate embodiment, the biological fluids are clarified and the precipitated microvesicles are collected by filtration. In certain embodiments, filtration of either the biological fluid, and/or the precipitated microvesicles required the application of an external force. The external force may be gravity, either normal gravity or centrifugal force. Alternatively, the external force may be suction.

In one embodiment, the present embodiment provides an apparatus to facilitate the clarification of the biological fluid by filtration. In one embodiment, the present disclosure provides an apparatus to facilitate collection of the precipitated microvesicles by filtration. In one embodiment, the present disclosure provides an apparatus that facilitates the clarification of the biological fluid and the collection of the precipitated microvesicles by filtration. In one embodiment, the apparatus also washes the microvesicles.

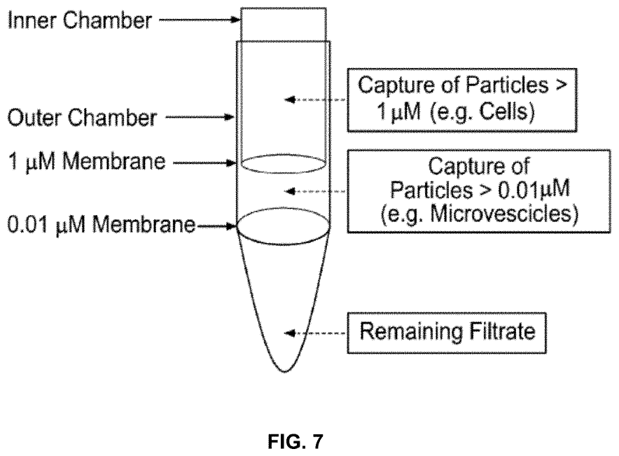

In one embodiment, the apparatus is the apparatus shown in FIG. 7. In this embodiment, the biological fluid is added to the inner chamber. The inner chamber has a first filter with a pore size that enables the microvesicles to pass, while retaining any particle with a size greater than a microvesicle in the inner chamber. In one embodiment, the pore size of the filter of the inner chamber is 1 μm. In this embodiment, when the biological fluid passed from the inner chamber through the filter, particles greater than 1 μm are retained in the inner chamber, and all other particles collect in the region between the bottom of the inner chamber and a second filter.

The second filter has a pore size that does not allow microvesicles to pass. In one embodiment, the pore size of the second filter of the inner chamber is 0.01 μm. In this embodiment, when the biological fluid passed through the second filter, the microvesicles are retained in the region between the bottom of the inner chamber and the second filter, and all remaining particles and fluid collect in the bottom of the apparatus.

One of ordinary skill in the art can readily appreciate that the apparatus can have more than two filters, of varying pore sizes to select for microvesicles of desired sizes, for example.

In one embodiment, a precipitating agent is added to the biological fluid in the inner chamber. In one embodiment, a precipitating agent is added to the filtrate after it has passed through the first filter. The filter membranes utilized by the apparatus described herein may be made from any suitable material, provided the filter membrane does not react with the biological fluid, or bind with components within the biological fluid. For example, the filter membranes may be made from a low bind material, such as, for example, polyethersulfone, nylon6, polytetrafluoroethylene, polypropylene, zeta modified glass microfiber, cellulose nitrate, cellulose acetate, polyvinylidene fluoride, regenerated cellulose.

In one embodiment, the microvesicles are isolated from culture medium conditioned using bone marrow derived stem cells. In one embodiment, the microvesicles are isolated from culture medium conditioned using bone marrow derived stem according to the methods of U.S. Pat. No. 10,500,231, incorporated by reference herein in its entirety.

### Characterization of Microvesicles

In one embodiment, the microvesicles have a size of about 2 nm to about 5000 nm as determined by electron microscopy. In an alternate embodiment, the microvesicles described herein have a size of about 2 nm to about 1000 nm as determined by electron microscopy. In an alternate embodiment, the microvesicles described herein have a size of about 2 nm to about 500 nm as determined by electron microscopy. In an alternate embodiment, the microvesicles described herein have a size of about 2 nm to about 400 nm as determined by electron microscopy. In an alternate embodiment, the microvesicles described herein have a size of about 2 nm to about 300 nm as determined by electron microscopy. In an alternate embodiment, the microvesicles described herein have a size of about 2 nm to about 200 nm as determined by electron microscopy. In an alternate embodiment, the microvesicles described herein have a size of about 2 nm to about 100 nm as determined by electron microscopy. In an alternate embodiment, the microvesicles described herein have a size of about 2 nm to about 50 nm as determined by electron microscopy. In an alternate embodiment, the microvesicles described herein have a size of about 2 nm to about 20 nm as determined by electron microscopy. In an alternate embodiment, the microvesicles described herein have a size of about 2 nm to about 10 nm as determined by electron microscopy.

In one embodiment, the microvesicles described herein have a molecular weight of at least 100 kDa.

Microvesicles isolated according to the methods described herein may be used for therapies. Alternatively, the microvesicles described herein may be used to alter or engineer cells or tissues. In the case where the microvesicles described herein are used to alter or engineer cells or tissues, the microvesicles may be loaded, labeled with RNA, DNA, lipids, carbohydrates, protein, drugs, small molecules, metabolites, or combinations thereof, that will alter or engineer a cell or tissue. Alternatively, the microvesicles may be isolated from cells or tissues that express and/or contain the RNA, DNA, lipids, carbohydrates, protein, drugs, small molecules, metabolites, or combinations thereof.

In some embodiments, the microvesicles have the characteristics of the microvesicles described in U.S. Pat. No. 10,500,231, incorporated by reference herein in its entirety.

In some embodiments, the microvesicles described herein have borders that are smoother, uncorrugated and appear more “intact” when compared to microvesicles isolated by ultracentrifuge isolation.

In some embodiments, the microvesicles described herein comprise exosomal markers including, but not limited to: HSP 70 and CD63. In some embodiments the exosomes contain the transcription factor STAT3 In some embodiments the exosomes contain the activated phosphorylated form phospho-STAT3.

In some embodiments, the microvesicles described herein promote fibroblast proliferation and migration as described in U.S. Pat. No. 10,500,231, incorporated by reference herein in its entirety.

In some embodiments, the microvesicles described herein demonstrate uptake into cells as described in U.S. Pat. No. 10,500,231, incorporated by reference herein in its entirety.

### Pharmaceutical Compositions

The microvesicles described herein can be used as a therapy to treat a disease.

In one embodiment, the microvesicles described herein are used to deliver molecules to cells. The delivery of molecules may be useful in treating or preventing a disease. In one embodiment, the delivery is according to the methods described in PCT Application WO04014954A1, incorporated by reference herein in its entirety. In an alternate embodiment, the delivery is according to the methods described in PCT Application WO2007126386A1, incorporated by reference herein in its entirety. In an alternate embodiment, the delivery is according to the methods described in PCT Application WO2009115561A1, incorporated by reference herein in its entirety. In an alternate embodiment, the delivery is according to the methods described in PCT Application WO2010119256A1, incorporated by reference herein in its entirety.

In one embodiment, the present disclosure provides an isolated preparation of microvesicles that can promote functional regeneration and organization of complex tissue structures. In one embodiment the present disclosure provides an isolated preparation of microvesicles that can regenerate hematopoietic tissue in a patient with aplastic anemia. In one embodiment the present disclosure provides an isolated preparation of microvesicles that can regenerate at least one tissue in a patient with diseased, damages or missing skin selected from the group consisting of: epithelial tissue, stromal tissue, nerve tissue, vascular tissue and adnexal structures. In one embodiment, the present disclosure provides an isolated preparation of microvesicles that can regenerate tissue and/or cells from all three germ layers.

In one embodiment, the present disclosure provides an isolated preparation of microvesicles that is used to modulate the immune system of a patient.

In one embodiment, the present disclosure provides an isolated preparation of microvesicles that enhances the survival of tissue or cells that is transplanted into a patient. In one embodiment, the patient is treated with the isolated preparation of microvesicles prior to receiving the transplanted tissue or cells. In an alternate embodiment, the patient is treated with the isolated preparation of microvesicles after receiving the transplanted tissue or cells. In an alternate embodiment, the tissue or cells is treated with the isolated preparation of microvesicles. In one embodiment, the tissue or cells is treated with the isolated preparation of microvesicles prior to transplantation.

In one embodiment, the patient receives a transplant of tissue or cells wherein the tissue or cells deliver microvesicles to the patient. In some embodiments, the transplanted tissue or cells are mesenchymal stem cells. In some embodiments, the mesenchymal stem cells are bone marrow mesenchymal stem cells.

In one embodiment, the present disclosure provides an isolated preparation of microvesicles containing at least one molecule selected from the group consisting of RNA, DNA, lipid, carbohydrate, metabolite, protein, and combination thereof from a host cell. In one embodiment, the host cell is engineered to express at least one molecule selected from the group consisting of RNA, DNA, lipid, carbohydrate, metabolite, protein, and combination thereof. In one embodiment, the isolated preparation of microvesicles containing at least one molecule selected from the group consisting of RNA, DNA, lipid, carbohydrate, metabolite, protein, and combination thereof from a host cell is used as a therapeutic agent.

For therapeutic use, in some embodiments, MVs are combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, EV compositions described herein can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of EV composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody molecule components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

EV compositions can also include a buffer or a pH adjusting agent; typically, the buffer is a salt prepared from an organic acid or base. Representative buffers include organic acid salts such as salts of citric acid, acetic acid, ascorbic acid, gluconic acid, carbonic acid, tartaric acid, succinic acid, or phthalic acid; Tris, tromethamine hydrochloride, or phosphate buffers.

Additionally, EV compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the antibody molecule compositions according to the invention are known in the art, e.g., as listed in “Remington: The Science & Practice of Pharmacy,” 19th ed., Williams & Williams, (1995), and in the “Physician's Desk Reference,” 52nd ed., Medical Economics, Montvale, N.J. (1998). Preferred carrier or excipient materials are carbohydrates (e.g., saccharides and alditols) and buffers (e.g., citrate) or polymeric agents.

The present disclosure provides for stable compositions, comprising MVs in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, or 1.0%), 0.1-3% benzyl alcohol (e.g., 0.5, 0.9, 1.1., 1.5, 1.9, 2.0, or 2.5%), 0.001-0.5% thimerosal (e.g., 0.005 or 0.01%), 0.001-2.0% phenol (e.g., 0.05, 0.25, 0.28, 0.5, 0.9, or 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, or 1.0%), and the like.

Pharmaceutical compositions containing MVs as disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for MVs is topical administration. Useful formulations can be prepared by methods known in the pharmaceutical art. For example, see Remington's Pharmaceutical Sciences (1990) supra. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as EDTA; buffers such as acetates, citrates or phosphates; and agents for the adjustment of tonicity such as sodium chloride or dextrose.

The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

The compositions of this invention may be in a variety of forms. These include, for example, liquid, semi-solid and solid dosage forms, such as liquid solutions (e.g., injectable and infusible solutions), dispersions or suspensions, and liposomes. The preferred form depends on the intended mode of administration and therapeutic application. Typical preferred compositions are in the form of injectable or infusible solutions. The preferred mode of administration is parenteral (e.g., intravenous, subcutaneous, intraocular, intraperitoneal, intramuscular). In a preferred embodiment, the preparation is administered by intravenous infusion or injection. In another preferred embodiment, the preparation is administered by intramuscular or subcutaneous injection.

The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

The present disclosure provides a kit, comprising packaging material and at least one vial comprising a solution of MVs with the prescribed buffers and/or preservatives, optionally in an aqueous diluent. The aqueous diluent optionally further comprises a pharmaceutically acceptable preservative. Preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer can be added to provide improved pH control. The formulations can cover a wide range of pHs, such as from about pH 4.0 to about pH 10.0, from about pH 5.0 to about pH 9.0, or about pH 6.0 to about pH 8.0.

Other additives, such as a pharmaceutically acceptable solubilizers like TWEEN 20 (polyoxyethylene (20) sorbitan monolaurate), TWEEN 40 (polyoxyethylene (20) sorbitan monopalmitate), TWEEN 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

Various delivery systems can be used to administer MVs to a subject. In certain exemplary embodiments, administration of MVs is topical, optionally with the addition of a dressing, bandage, medical tape, pad, gauze or the like. Suitable dressings to aid in topical delivery are well-known in the art and are commercially available. In other embodiments, MVs are administered by pulmonary delivery, e.g., by intranasal administration, or by oral inhalative administration. Pulmonary delivery may be achieved via a syringe or an inhaler device (e.g., a nebulizer, a pressurized metered-dose inhaler, a multi-dose liquid inhaler, a thermal vaporization aerosol device, a dry powder inhaler or the like). Suitable methods for pulmonary delivery are well-known in the art and are commercially available.

Any of the formulations described above can be stored in a liquid or frozen form and can be optionally subjected to a preservation process.

In certain exemplary embodiments of the invention, EVs described herein are used to deliver one or more bioactive agents to a target cell. The term “bioactive agent” is intended to include, but is not limited to, proteins (e.g., non-membrane-bound proteins), peptides (e.g., non-membrane-bound peptides), transcription factors, nucleic acids and the like, that are expressed in a cell and/or in a cellular fluid and are added during the purification and/or preparation of EVs described herein, and/or pharmaceutical compounds, proteins (e.g., non-membrane-bound proteins), peptides (e.g., non-membrane-bound peptides), transcription factors, nucleic acids and the like, that EVs described herein are exposed to during one or more purification and/or preparation steps described herein. In certain embodiments, a bioactive agent is a collagen VII protein, a collagen VII mRNA, a STAT3 signaling activator (e.g., an interferon, epidermal growth factor, interleukin-5, interleukin-6, a MAP kinase, a c-src non-receptor tyrosine kinase or another molecule that phosphorylates and/or otherwise activates STAT3) and/or a canonical Wnt activator (see, e.g., McBride et al. (2017) Transgenic expression of a canonical Wnt inhibitor, kallistatin, is associated with decreased circulating CD19+B lymphocytes in the peripheral blood. International Journal of Hematology, 1-10. DOI: 10.1007/s12185-017-2205-5, incorporated herein by reference in its entirety). In some embodiments, the bioactive agent is a type IV collagen protein and/or a type IV collagen mRNA. In some embodiments, the bioactive agent is a plectin protein and/or a plectin mRNA. In some embodiments, the bioactive agent is a bullous pemphigoid antigen 1 protein and/or a bullous pemphigoid antigen 1 mRNA. In some embodiments, the bioactive agent is a keratin 1 protein and/or a keratin 1 mRNA. In some embodiments, the bioactive agent is a hSPCA1 protein and/or a hSPCA1 mRNA. In some embodiments, the bioactive agent is a lysosomal trafficking regulator protein and/or a lysosomal trafficking regulator mRNA. In some embodiments, the bioactive agent is a serine-protein kinase ATM protein and/or a serine-protein kinase ATM mRNA. In some embodiments, the bioactive agent is a tuberin protein and/or a tuberin mRNA. In some embodiments, the bioactive agent is a FOXM1A protein and/or a FOXM1A mRNA. In other embodiments, a bioactive agent is one or more pharmaceutical compounds known in the art.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EXAMPLES

### Example 1: Analysis of the Secretome of Bone Marrow Mesenchymal Stem Cells

### Overview

In order to identify proteins in the bone marrow-derived mesenchymal stem cell secretome that are relevant to cutaneous structure and disease, bone marrow aspirates were obtained from four healthy donors. BM-MSCs from each donor were isolated and separately cultured, followed by incubation in serum-free culture media to allow for production and collection of the BM-MSC secretome. Extracellular vesicles were isolated for analysis. Mass spectrometry and Proteome Discoverer was used to identify proteins secreted by each of the four healthy donors. Functional categorization of proteins was classified using UniProt Knowledgebase.

### Methods

Bone marrow donors: Collection of primary human donor bone marrow was under approval of University of Miami Institutional Review Board (IRB) and in accordance of policies of the Interdisciplinary Stem Cell Institute. All experiments were performed in accordance with relevant guidelines and regulations and complied with the Declaration of Helsinki. Informed consent was obtained for all human subjects and permission was given by all 4 human subjects to publish results derived from the tissues and cells and, if necessary, to publish any identifying information, including images. The human donors of bone marrow were: 33 year old male (donor 1), 33 year old female (donor 2), 28 year old female (donor 3), and 28 year old male (donor 4). As is standard for bone marrow donors at the Interdisciplinary Stem Cell Institute, all 4 donors tested negative for anti-HIV-1/HIV-2, anti-HTLV I/II, anti-HCV, HIV-1 nucleic acid test, HCV nucleic acid test, HBsAg, anti-HBc (IgG and IgM), anti-CMV, WNV nucleic acid, T. cruzi ELISA (Chagas), RPR for syphilis, and had no clinical/history/laboratory evidence to suggest Creutzfeldt-Jakob disease. Bone marrow (approximately 80 mL) was aspirated from the posterior iliac crests as per standard practice of the University of Miami Bone Marrow (BM) Transplant Programs. The marrow was aspirated into heparinized syringes and labeled syringes were transported at room temperature to the Good Manufacturing Practices (GMP) facility at the Interdisciplinary Stem Cell Institute at the University of Miami. Bone marrow (BM) was processed using Lymphocyte Separation Medium (LSM; specific gravity 1.077) to prepare the density-enriched, mononuclear cells (MNCs). Cells were diluted with Plasmalyte A or PBS buffer and layered onto LSM using conical tubes to isolate MNCs following established standardized operating procedures. The MNCs were washed with Plasmalyte A or PBS buffer containing 1% human serum albumin (HSA). The washed cells were sampled to determine the total number of viable nucleated cells. MSCs were initially cultured in alpha MEM media supplemented with 2 mM L-glutamine, 20% Fetal Bovine Serum (FBS), 100 units/ml penicillin, and 100 μg/ml streptomycin. The expansion was performed in flasks using a 37° C., 5% CO₂ humidified incubator. MSCs were detached from the culture vessels using trypsin exposure, passaged and cryopreserved at passage three prior to use in the following experiments. MSCs were verified in the GMP as viable, CD105⁺, CD45⁻ cells, sterile, mycoplasma-free and endotoxin-free.

Isolation of secretome and extracellular vesicles: Serum-free conditioned media was collected from each donor and extracellular vesicles (EVs) were isolated using ExoQuick-TC® ULTRA EV Isolation Kit for Tissue Culture Media (Cat #EQULTRA-20TC-1), according to manufacturer's instructions. Dot Blot was performed to verify extracellular vesicles were isolated without cellular contaminants (Exo-Check Exosome Antibody Arrays, Cat #EXORAY200A-4, Cat #EXORAY210A-8) according to manufacturer's instructions.

Processing of EV samples prior to mass spectrometry analysis: Lysing the EVs was completed as follows (all reagents from Sigma, unless otherwise stated). Isolated extracellular vesicles were centrifuged for 10 minutes at 2,000×g at 4° C. Samples were speed vacuumed dry until the sample was dry. Fifty μl of 20 mM Tris-2% SDS was added. The mixture was heated at 95° C. for 30 seconds, chilled for 30 seconds and cycled for a total of 5 minutes. Samples were sonicated for 1 minute. Proteins were precipitated with cold acetone. Samples were speed vacuumed until dry and resuspended in 100 μl ammonium bicarbonate. Eight μg of protein was added, centrifuged for 10 min. and speed vacuumed until sample was dry. Eight μl of 50 mM ammonium bicarbonate (pH 7.8) was added to the samples. Samples underwent denaturation with 15 μl of 10 M urea in 50 mM ammonium bicarbonate (pH 7.8). Samples were reduced using 2 μl of 125 DTT in 50 mM Ammonium Bicarbonate (pH 7.8). Samples were incubated for 1 hour at room temperature. Samples underwent alkylation with 5 μl of [90 mM Iodoacetamide in 50 mM Ammonium Bicarbonate, pH (7.8), and incubated in room temperature for 30 min. Samples were quenched with 3.33 μl of 125 mM DTT in 50 mM ammonium bicarbonate (pH 7.8). Samples were incubated at room temperature for 1 hour in the dark. Ammonium bicarbonate (50 mM) was added to dilute urea to 1 molar concentration. Samples were digested with trypsin corresponding to 1:30 w/w enzyme to protein and incubated overnight at 37° C. for 18 hours. Formic acid (50%) was added to stop trypsin reaction (5:100 v/v formic acid to sample). Samples were desalted using the Pierce C18 Spin Tips (Thermo Scientific). Trifluoroacetic acid (TFA) (2.5%) was added to sample to adjust TFA concentration to 0.05%; pH of less than 4 was verified. C18 Spin Tips were used were placed into a spin adapter and tip was wetted with 0.1% TFA in 80% acetonitrile (ACN), and centrifuged for 1 minute. After discarding the flow through, the sample was added to C18 spin tip and centrifuged at 1000×g for 1 minute; this process was repeated until all sample was passed through the C18 Spin Tip. The Spin Tip was then transferred to a fresh microcentrifuge tube. Sample was eluted by adding 20 μl of 0.1% TFA in 80% ACN and centrifuging at 1000×g for 1 minute; this step was repeated again to further elute sample. The sample was speed vacuumed to dry. The samples were reconstituted in 50 μL of 2% acetonitrile in LC-MS grade water with 0.1% formic acid prior to LC-MS/MS analysis.

High Performance Liquid Chromatography (HPLC) and Mass Spectrometry: The following methods were performed as previously described. (See Musada G R, Dvoriantchikova G, Myer C, Ivanov D, Bhattacharya S K, Hackam A S. The effect of extrinsic Wnt/beta-catenin signaling in Muller glia on retinal ganglion cell neurite growth. Dev Neurobiol 2020). In brief, reversed-phase chromatographic separation utilized an Easy-nLC 1000 system (Thermo) with an Acclaim PepMap RSLC 75 μm×15 cm, nanoViper column (Thermo). The solvents were LC-MS grade water and acetonitrile with 0.1% Formic Acid. Peptides were analyzed using a Q Exactive mass spectrometer (Thermo) with a heated electrospray ionization source (HESI) operating in positive ion mode. Protein identifications from MS/MS data utilized the Proteome Discoverer 2.2 software (Thermo Fisher Scientific) using Sequest HT search engines. The data was searched against the Homo sapiens entries in Uniprot protein sequence database. The search parameters included: precursor mass tolerance 10 ppm and 0.02 Da for fragments, 2 missed trypsin cleavages, oxidation (Met) and acetylation (protein N-term) as variable modifications, carbamidomethylation (Cys) as a static modification. Percolator PSM validation was used with the following parameters: strict false discover rate of 0.01, relaxed FDR of 0.1, maximum ΔCn of 0.05, validation based on q-value. We obtained the high confidence peptides and filtered out the low and medium confidence peptides.

### Summary of the Results

The secretome of donors 1 through 4 contained 3373, 3457, 3523, and 3267 uniquely identifiable protein products, respectively. There were 636 common proteins detected in the secretome of all four healthy donors. Proteins were categorized based on cellular components, biological processes, ligand functions, and disease correlations. Highlighted here is the discovery of proteins detected in the secretome of all four donors, especially those relevant to skin homeostasis and cutaneous disease. These proteins included basement membrane proteins type IV collagen (forms the lamina densa), type VII collagen (forms anchoring fibrils and mutated in dystrophic epidermolysis bullosa), plectin and bullous pemphigoid antigen 1 (both part of the hemidesmosome and mutated in forms of epidermolysis bullosa simplex), keratinocyte-related proteins such as epiplakin, keratin 1, soluble e-cadherin, and, interestingly, proteins traditionally not reported to be part of the secretome: calcium transporting ATPase hSPCA1 (the latter encoded by ATP2C1, mutated in benign familial pemphigus/Hailey-Hailey disease), tuberin (TSC2, mutated in tuberous sclerosis), lysosomal trafficking regulator (LYST, mutated in Chediak-Higashi syndrome), and the serine protein kinase ATM (mutated in Ataxia-Telangiectasia).

This example demonstrates that the human bone marrow mesenchymal stem cell secretome contains important proteins involved in cutaneous homeostasis and disease. The secretome of the bone marrow mesenchymal stem contains common proteins among donors. These proteins are important in basement membrane structure, and some code for proteins mutated in genodermatoses.

### Detailed Results

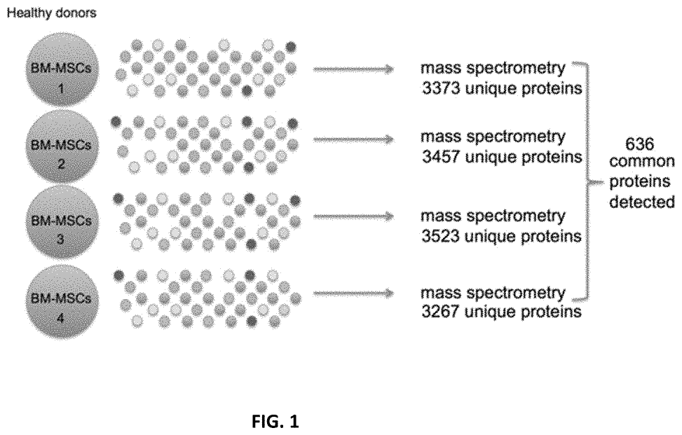

The BM-MSC secretome from each bone marrow donor 1 through 4 included: 3398, 3486, 3566, and 3293 uniquely identifiable proteins, respectively. As shown in FIG. 1, in total, this represented 636 unique proteins in total from the BM-MSC secretome of the four bone marrow donors. Proteins were categorized based on known functionality assigned in the UniProt Knowledgebase (uniprot.org). The entire list of functional categorization can be found by searching the following on UniProt.org and selecting keywords (“yourlist:M20200525A94466D2655679D1FD8953E075198DA8E760DF0”).

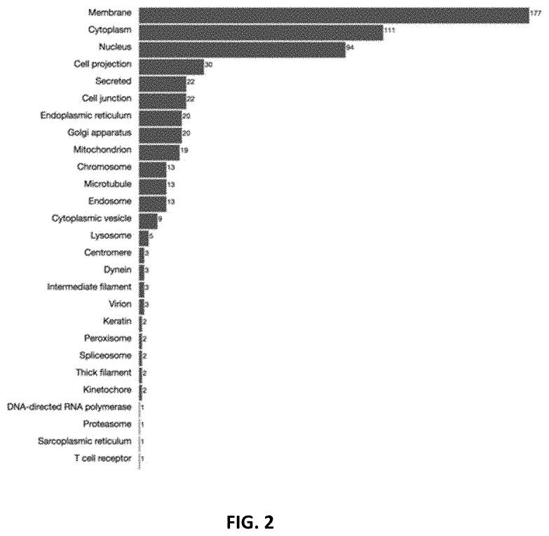

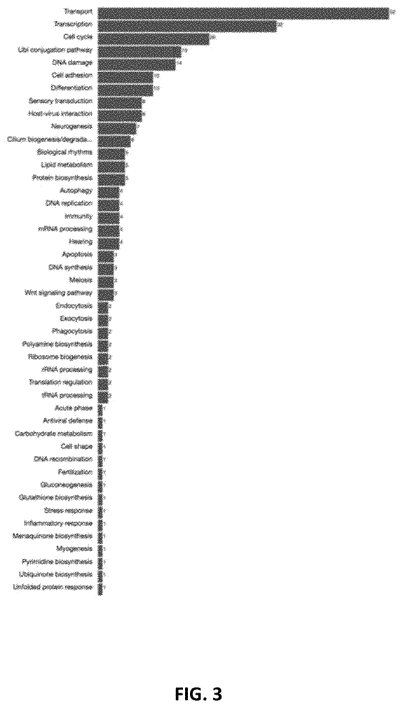

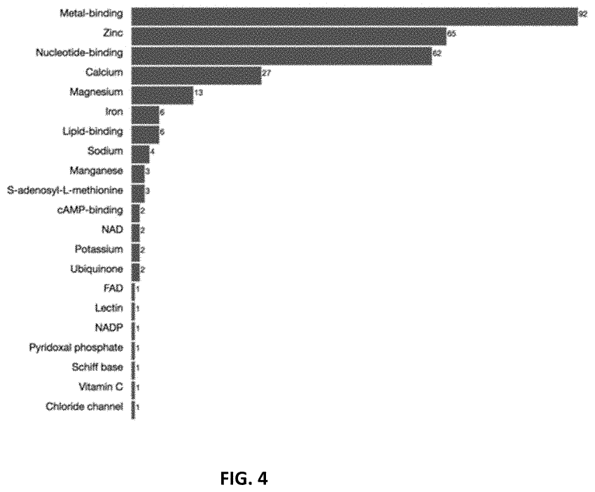

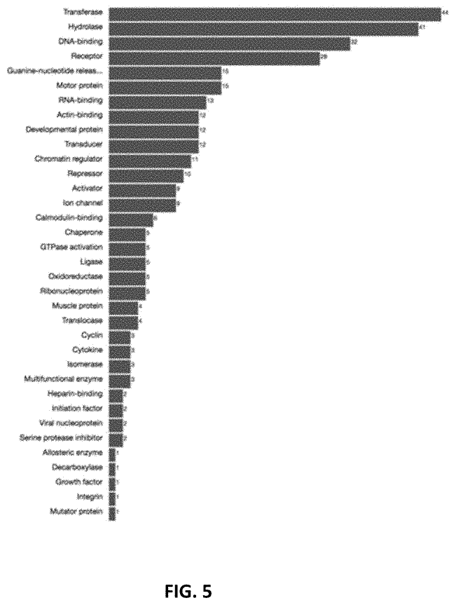

As shown in FIG. 2, in the category of cellular components common among all 4 donors most classifiable proteins were associated with the cell membrane (177 proteins). The next most abundant were proteins associated with the cell cytoplasm (111 proteins). Interestingly, the third most common was proteins associated with the nucleus (94 proteins). Proteins associated with cell projection were the fourth most abundant (30 proteins). Proteins traditionally thought of as “secreted” were the fifth most abundant category (22 proteins). The full list of cellular component categories are presented in FIG. 2. As shown in FIG. 3, when examining the proteins' biological processes' common among all 4 donors, the most prevalent category of proteins was associated with the function of transport (52 proteins). The second most prevalent category were proteins associated with transcription (32 proteins). Third most prevalent were proteins associated with cell cycle regulation (20 proteins). Ubiquitylation-conjugation pathway associated proteins were the fourth most common (15 proteins). Proteins involved in DNA damage regulation (14 proteins), cell adhesion (10 proteins), and cell differentiation (10 proteins) were the next most prevalent categories. The other biologic processes categories are shown in FIG. 3. As shown in FIG. 4, in the category of ligand-binding, many of the proteins common among all 4 donors were classified as metal-binding proteins (92 proteins). Specifically, most proteins appear to bind to zinc (65 proteins). Nucleotide-binding proteins were also prevalent (62 proteins). Calcium-binding proteins were common (27 proteins). Proteins that bind magnesium (13 proteins), iron (6 proteins), and lipids (6) were also detected. FIG. 4 provides further ligand-binding groups. As shown in FIG. 5, in terms of molecular function, most proteins common among all 4 donors were grouped as transferases (44 proteins), hydrolases (41 proteins), DNA-binding (32 proteins), receptors (28), guanine-nucleotide releasing factors (15 proteins), motor proteins (15 proteins), RNA-binding proteins (13 proteins), actin-binding proteins (12 proteins), developmental proteins (12 proteins), transducers (12 proteins), and chromatin regulators (11 proteins).

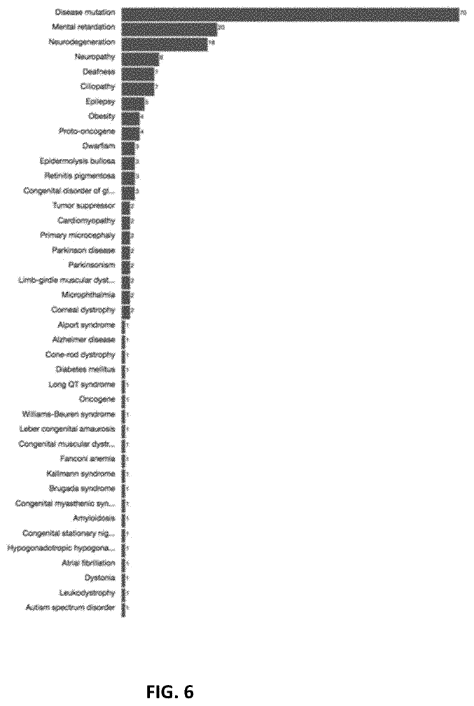

As shown in FIG. 6, many proteins common among all 4 donors were the products of genes mutated in various diseases (70 proteins). The most prevalent groups included proteins implicated in mental retardation (20 proteins) and neurodegeneration (18 proteins). Deafness (7 proteins), ciliopathy (7 proteins), epilepsy (5 proteins), obesity (4 proteins), dwarfism (3 proteins), epidermolysis bullosa (3 proteins), and retinitis pigmentosa (3 proteins). Proteins common among all 4 donors that were particularly important in the cutaneous structure and function were found. As shown in table 1 below, proteins that are significantly implicated in skin basement membrane, the hemidesmosome, and keratinocyte homeostasis were identified. Among these proteins were type IV collagen (which forms the lamina densa of the basement membrane of the skin), type VII collagen (which forms anchoring fibrils and mutated in both autosomal recessive and autosomal dominant dystrophic epidermolysis bullosa), plectin (which is mutated in epidermolysis bullosa simplex with pyloric atresia and muscular dystrophy) and bullous pemphigoid antigen 1 (which is both part of hemidesmosome and mutated in a form of autosomal recessive epidermolysis bullosa simplex). Keratinocyte-related proteins included epiplakin, keratin 1, soluble e-cadherin (Table 1), and calcium transporting ATPase hSPCA1 (the latter encoded by ATP2C1 and mutated in benign familial pemphigus/Hailey-Hailey disease) (Table 2). Furthermore, proteins involved in neurocutaneous disorders, such as tuberin (TSC2, mutated in tuberous sclerosis); and immune system-related proteins that result in cutaneous phenotypes, such as lysosomal trafficking regulator (LYST, mutated in Chediak-Higashi syndrome), and the serine protein kinase ATM (mutated in Ataxia-Telangiectasia) (Table 2).

TABLE 1
Selected proteins from secretome of all 4 BM-MSC donors involved
in basement membrane and hemidesmosomal structure

| | Gene | | Involvement in |
|---|---|---|---|
| Protein name(s) | name(s) | Function(s) | disease(s) |
| Collagen alpha- | COL4A4 | Type IV collagen | Deep burns result in |
| 4(IV) chain | | is the major | loss of type IV |
| | | structural | collagen. Effective |
| | | component of the | non-scarring |
| | | cutaneous and | regeneration relies |
| | | glomerular | on re-establishment |
| | | basement | of type IV collagen |
| | | membrane, forming | in the regenerated |
| | | a meshwork | basement |
| | | together with | membrane. |
| | | laminins, | Alport syndrome 2, |
| | | proteoglycans and | autosomal recessive; |
| | | entactin/nidogen. | a syndrome |
| | | | characterized by |
| | | | progressive |
| | | | glomerulonephritis, |
| | | | glomerular |
| | | | basement |
| | | | membrane defects, |
| | | | renal failure, |
| | | | sensorineural |
| | | | deafness and |
| | | | specific eye |
| | | | abnormalities |
| | | | (lenticonous and |
| | | | macular flecks). The |
| | | | disorder shows |
| | | | considerable |
| | | | heterogeneity in that |
| | | | families differ in the |
| | | | age of end-stage |
| | | | renal disease and the |
| | | | occurrence of |
| | | | deafness. Loss of |
| | | | protein can result in |
| | | | hematuria, benign |
| | | | familial. An |
| | | | autosomal dominant |
| | | | condition |
| | | | characterized by |
| | | | non-progressive |
| | | | isolated microscopic |
| | | | hematuria that does |
| | | | not result in renal |
| | | | failure. It is |
| | | | characterized |
| | | | pathologically by |
| | | | thinning of the |
| | | | glomerular |
| | | | basement |
| | | | membrane. |
| Collagen alpha- | COL7A1 | Stratified squamous | Epidermolysis |
| 1(VII) chain | | epithelial basement | bullosa acquisita |
| (Long-chain | | membrane protein | (EBA) is an |
| collagen) | | that forms | autoimmune |
| | | anchoring fibrils | acquired blistering |
| | | which may | skin disease |
| | | contribute to | resulting from |
| | | epithelial basement | autoantibodies to |
| | | membrane | type VII collagen. |
| | | organization and | Epidermolysis |
| | | adherence by | bullosa dystrophica, |
| | | interacting with | autosomal dominant |
| | | extracellular matrix | (DDEB). A group of |
| | | (ECM) proteins | autosomal dominant |
| | | such as type IV | blistering skin |
| | | collagen | diseases |
| | | | characterized by |
| | | | tissue separation |
| | | | which occurs below |
| | | | the dermal- |
| | | | epidermal basement |
| | | | membrane at the |
| | | | level of the |
| | | | anchoring fibrils. |
| | | | Various clinical |
| | | | types with different |
| | | | severity are |
| | | | recognized, ranging |
| | | | from severe |
| | | | mutilating forms to |
| | | | mild forms with |
| | | | limited and |
| | | | localized scarring, |
| | | | and less frequent |
| | | | extracutaneous |
| | | | manifestations. |
| | | | Epidermolysis |
| | | | bullosa dystrophica, |
| | | | autosomal recessive |
| | | | (RDEB). A group of |
| | | | autosomal recessive |
| | | | blistering skin |
| | | | diseases |
| | | | characterized by |
| | | | tissue separation |
| | | | which occurs below |
| | | | the dermal- |
| | | | epidermal basement |
| | | | membrane at the |
| | | | level of the |
| | | | anchoring fibrils. |
| | | | Various clinical |
| | | | types with different |
| | | | severity are |
| | | | recognized, ranging |
| | | | from severe |
| | | | mutilating forms, |
| | | | such as |
| | | | epidermolysis |
| | | | bullosa dystrophica |
| | | | Hallopeau-Siemens |
| | | | type, to mild forms |
| | | | with limited |
| | | | localized scarring |
| | | | and less frequent |
| | | | extracutaneous |
| | | | manifestations. Mild |
| | | | forms include |
| | | | epidermolysis |
| | | | bullosa mitis and |
| | | | epidermolysis |
| | | | bullosa localisata. |
| | | | Transient bullous |
| | | | dermolysis of the |
| | | | newborn (TBDN). |
| | | | TBDN is a neonatal |
| | | | form of dystrophic |
| | | | epidermolysis |
| | | | bullosa |
| | | | characterized by |
| | | | sub-epidermal |
| | | | blisters, reduced or |
| | | | abnormal anchoring |
| | | | fibrils at the dermo- |
| | | | epidermal junction, |
| | | | and electron-dense |
| | | | inclusions in |
| | | | keratinocytes. |
| | | | TBDN heals |
| | | | spontaneously or |
| | | | strongly improves |
| | | | within the first |
| | | | months and years of |
| | | | life. |
| | | | Epidermolysis |
| | | | bullosa dystrophica, |
| | | | pretibial type (PR- |
| | | | DEB). A form of |
| | | | dystrophic |
| | | | epidermolysis |
| | | | bullosa |
| | | | characterized by |
| | | | pretibial blisters that |
| | | | develop into |
| | | | prurigo-like |
| | | | hyperkeratotic |
| | | | lesions. It |
| | | | predominantly |
| | | | affects the pretibial |
| | | | areas, sparing the |
| | | | knees and other |
| | | | parts of the skin. |
| | | | Other clinical |
| | | | features include nail |
| | | | dystrophy, |
| | | | albopapuloid skin |
| | | | lesions, and |
| | | | hypertrophic scars |
| | | | without pretibial |
| | | | predominance. The |
| | | | phenotype shows |
| | | | considerable |
| | | | interindividual |
| | | | variability. |
| | | | Inheritance is |
| | | | autosomal |
| | | | dominant. |
| | | | Epidermolysis |
| | | | bullosa dystrophica, |
| | | | Bart type (B-DEB): |
| | | | An autosomal |
| | | | dominant form of |
| | | | dystrophic |
| | | | epidermolysis |
| | | | bullosa |
| | | | characterized by |
| | | | congenital localized |
| | | | absence of skin, |
| | | | skin fragility and |
| | | | deformity of nails. |
| | | | Epidermolysis |
| | | | bullosa pruriginosa. |
| | | | A distinct clinical |
| | | | subtype of |
| | | | epidermolysis |
| | | | bullosa dystrophica. |
| | | | It is characterized |
| | | | by skin fragility, |
| | | | blistering, scar |
| | | | formation, intense |
| | | | pruritus and |
| | | | excoriated prurigo |
| | | | nodules. Onset is in |
| | | | early childhood, but |
| | | | in some cases is |
| | | | delayed until the |
| | | | second or third |
| | | | decade of life. |
| | | | Inheritance can be |
| | | | autosomal dominant |
| | | | or recessive. |
| | | | Nail disorder, non- |
| | | | syndromic |
| | | | congenital, 8. A nail |
| | | | disorder |
| | | | characterized by |
| | | | isolated toenail |
| | | | dystrophy. The nail |
| | | | changes are most |
| | | | severe in the great |
| | | | toes and consist of |
| | | | the nail plate being |
| | | | buried in the nail |
| | | | bed with a deformed |
| | | | and narrow free |
| | | | edge. |
| | | | Epidermolysis |
| | | | bullosa dystrophica, |
| | | | with subcorneal |
| | | | cleavage. A bullous |
| | | | skin disorder with |
| | | | variable sized clefts |
| | | | just beneath the |
| | | | level of the stratum |
| | | | corneum. Clinical |
| | | | features include |
| | | | blisters, milia, |
| | | | atrophic scarring, |
| | | | nail dystrophy, and |
| | | | oral and |
| | | | conjunctival |
| | | | involvement, as |
| | | | seen in dystrophic |
| | | | epidermolysis |
| | | | bullosa. |
| Plectin (PCN) | PLEC1 | Interlinks | Epidermolysis |
| (PLTN) (Hemi- | | intermediate | bullosa simplex |
| desmosomal | | filaments with | with pyloric atresia. |
| protein 1) | | microtubules and | Autosomal recessive |
| (HD1) | | microfilaments and | genodermatosis |
| (Plectin-1) | | anchors | characterized by |
| | | intermediate | severe skin |
| | | filaments to | blistering at birth |
| | | desmosomes or | and congenital |
| | | hemidesmosomes. | pyloric atresia. |
| | | Could also bind | Death usually |
| | | muscle proteins | occurs in infancy. |
| | | such as actin to | Epidermolysis |
| | | membrane | bullosa simplex, |
| | | complexes in | with muscular |
| | | muscle. May be | dystrophy (MD- |
| | | involved not only in | EBS). A form of |
| | | the filaments | epidermolysis |
| | | network, but also in | bullosa |
| | | the regulation of | characterized by the |
| | | their dynamics. | association of blister |
| | | Structural | formation at the |
| | | component of | level of the |
| | | muscle. Isoform 9 | hemidesmosome |
| | | plays a major role in | with late-onset |
| | | the maintenance of | muscular dystrophy. |
| | | myofiber integrity. | Epidermolysis |
| | | | bullosa simplex, |
| | | | Ogna type (O-EBS). |
| | | | A form of |
| | | | intraepidermal |
| | | | epidermolysis |
| | | | bullosa |
| | | | characterized by |
| | | | generalized skin |
| | | | bruising, skin |
| | | | fragility with non- |
| | | | scarring blistering |
| | | | and small |
| | | | hemorrhagic blisters |
| | | | on hands. At the |
| | | | ultrastructural level, |
| | | | it is differentiated |
| | | | from classical cases |
| | | | of K-EBS, WC-EBS |
| | | | and DM-EBS, by |
| | | | the occurrence of |
| | | | blisters originating |
| | | | in basal cells above |
| | | | hemidesmosomes, |
| | | | and abnormal |
| | | | hemidesmosome |
| | | | intracellular |
| | | | attachment plates. |
| | | | Muscular dystrophy, |
| | | | limb-girdle, |
| | | | autosomal recessive |
| | | | 17. A form of limb- |
| | | | girdle muscular |
| | | | dystrophy |
| | | | characterized by |
| | | | early childhood |
| | | | onset of proximal |
| | | | muscle weakness. |
| | | | Limb-girdle |
| | | | muscular |
| | | | dystrophies are |
| | | | characterized by |
| | | | proximal weakness, |
| | | | weakness of the hip |
| | | | and shoulder girdles |
| | | | and prominent |
| | | | asymmetrical |
| | | | quadriceps femoris |
| | | | and biceps brachii |
| | | | atrophy. |
| | | | Epidermolysis |
| | | | bullosa simplex |
| | | | with nail dystrophy |
| | | | (EBSND). A form |
| | | | of epidermolysis |
| | | | bullosa, a |
| | | | dermatologic |
| | | | disorder |
| | | | characterized by |
| | | | skin blistering. |
| | | | EBSND patients |
| | | | also manifest nail |
| | | | dystrophy. |
| Dystonin (230 | DST, | Cytoskeletal linker | Epidermolysis |
| kDa bullous | BP230, | protein. Acts as an | bullosa simplex, |
| pemphigoid | BPAG1 | integrator of | autosomal recessive |
| antigen) | | intermediate | 2 (EBSB2). A form |
| (230/240 | | filaments, actin and | of epidermolysis |
| kDa bullous | | microtubule | bullosa, a |
| pemphigoid | | cytoskeleton | dermatologic |
| antigen) | | networks. Required | disorder |
| (Bullous | | for anchoring either | characterized by |
| pemphigoid | | intermediate | localized blistering |
| antigen 1) | | filaments to the | on the dorsal, lateral |
| (BPA) (Bullous | | actin cytoskeleton in | and plantar surfaces |
| pemphigoid | | neural and muscle | of the feet. EBSB2 |
| antigen) | | cells or keratin- | is characterized by |
| (Dystonia | | containing | trauma-induced |
| musculorum | | intermediate | blistering mainly |
| protein) | | filaments to | occurring on the feet |
| (Hemi- | | hemidesmosomes in | and ankles. |
| desmosomal | | epithelial cells. The | Ultrastructural |
| plaque | | proteins may self- | analysis of skin |
| protein) | | aggregate to form | biopsy shows |
| | | filaments or a two- | abnormal |
| | | dimensional mesh. | hemidesmosomes |
| | | Regulates the | with poorly formed |
| | | organization and | inner plaques. |
| | | stability of the | Neuropathy, |
| | | microtubule | hereditary sensory |
| | | network of sensory | and autonomic, 6 |
| | | neurons to allow | (HSAN6). A form |
| | | axonal transport. | of hereditary |
| | | Mediates docking of | sensory and |
| | | the dynein/dynactin | autonomic |
| | | motor complex to | neuropathy, a |
| | | vesicle cargos for | genetically and |
| | | retrograde axonal | clinically |
| | | transport. | heterogeneous |
| | | | group of disorders |
| | | | characterized by |
| | | | degeneration of |
| | | | dorsal root and |
| | | | autonomic ganglion |
| | | | cells, and by |
| | | | sensory and/or |
| | | | autonomic |
| | | | abnormalities. |
| | | | HSAN6 is a severe |
| | | | autosomal recessive |
| | | | disorder |
| | | | characterized by |
| | | | neonatal hypotonia, |
| | | | respiratory and |
| | | | feeding difficulties, |
| | | | lack of psychomotor |
| | | | development, and |
| | | | autonomic |
| | | | abnormalities |
| | | | including labile |
| | | | cardiovascular |
| | | | function, lack of |
| | | | corneal reflexes |
| | | | leading to corneal |
| | | | scarring, areflexia, |
| | | | and absent axonal |
| | | | flare response after |
| | | | intradermal |
| | | | histamine injection. |
| Epiplakin | EPPK1, | Cytoskeletal linker | Antigenic target in |
| (450 kDa | EPIPL | protein that | paraneoplastic |
| epidermal | | connects to | pemphigus |
| antigen) | | intermediate | |
| | | filaments and | |
| | | controls their | |
| | | reorganization in | |
| | | response to stress. | |
| | | In response to | |
| | | mechanical stress | |
| | | like wound healing, | |
| | | is associated with | |
| | | the machinery for | |
| | | cellular motility by | |
| | | slowing down | |
| | | keratinocyte | |
| | | migration and | |
| | | proliferation and | |
| | | accelerating keratin | |
| | | bundling in | |
| | | proliferating | |
| | | keratinocytes thus | |
| | | contributing to | |
| | | tissue architecture. | |
| | | However in wound | |
| | | healing in corneal | |
| | | epithelium also | |
| | | positively regulates | |
| | | cell differentiation | |
| | | and proliferation | |
| | | and negatively | |
| | | regulates migration | |
| | | thereby controlling | |
| | | corneal epithelium | |
| | | morphogenesis and | |
| | | integrity. In | |
| | | response to cellular | |
| | | stress, plays a role | |
| | | in keratin filament | |
| | | reorganization, | |
| | | probably by | |
| | | protecting keratin | |
| | | filaments against | |
| | | disruption. During | |
| | | liver and pancreas | |
| | | injuries, plays a | |
| | | protective role by | |
| | | chaperoning | |
| | | disease-induced | |
| | | intermediate | |
| | | filament | |
| | | reorganization. | |
| Keratin 1 | KRT1 | Keratins are a group | Defects in keratin 1 |
| | | of fibrous proteins | are a cause of |
| | | that form structural | epidermolytic |
| | | frameworks for | hyperkeratosis, also |
| | | keratinocytes to | known as bullous |
| | | make up the skin, | congenital |
| | | hair, and nails. | ichthyosiform |
| | | Keratin 1 partners | erythroderma, a |
| | | with either keratin 9 | hereditary skin |
| | | or 10 to form | disorder |
| | | heterodimer | characterized by |
| | | intermediate | intraepidermal |
| | | filaments, which | blistering, a marked |
| | | then assemble into | thickening of the |
| | | strong networks that | stratum corneum, |
| | | provide tensile | pigmentation of the |
| | | strength and | skin and erosions at |
| | | resiliency to the | sites of trauma |
| | | skin, protecting it | which are all present |
| | | from external | from birth. |
| | | damage. | |
| E-cadherin | CDH1 | Cadherins are | Associated with a |
| (soluble, | | calcium-dependent | variety of |
| fragment) | | cell adhesion | pathologies, |
| | | proteins vital in | including neoplasms |
| | | keratinocyte-to- | (gastric, ovarian, |
| | | keratinocyte | endometrial, breast), |
| | | adhesion | as it supports |
| | | | epithelial cell |
| | | | adherence (likely |
| | | | not a pathogenic |
| | | | factor). Recent |
| | | | studies have shown |
| | | | soluble e-cadherin |
| | | | to stimulate tumor |
| | | | angiogenesis, but |
| | | | stimulation of |
| | | | angiogenesis in a |
| | | | non-malignant |
| | | | tissue may be |
| | | | useful, such as in |
| | | | cutaneous repair and |
| | | | regeneration. |
| FOXM1A | FOXM1A | The transcription | Type 2 diabetes |
| | | factor Forkhead box | incidence increases |
| | | M1 (FOXM1) plays | with age, while β- |
| | | important roles in | cell replication |
| | | oncogenesis, | declines. |
| | | FOXM1A is one of | Furthermore, the |
| | | the FOXM1 | transcription factor |
| | | isoforms. | FoxM1 is required |
| | | | for β-cell replication |
| | | | in various situations, |
| | | | and its expression |
| | | | declines with age. |
| | | | Therefore, an |
| | | | increase in |
| | | | FOXM1A protein |
| | | | may have a role in |
| | | | alleviating the |
| | | | symptoms |
| | | | associated with |
| | | | diabetic foot ulcers. |

TABLE 2
Selected proteins from secretome of all 4 BM-MSC donors involved
in other genomic syndromes with cutaneous manifestations

| | | | Involvement in |
|---|---|---|---|
| Protein | Gene | Function | disease |
| Calcium- | ATP2C1 | This magnesium- | Benign familial |
| transporting | | dependent enzyme | pemphigus (Hailey- |
| ATPase (EC | | catalyzes the | Hailey disease) |
| 7.2.2.10) | | hydrolysis of ATP | |
| | | coupled with the | |
| | | transport of calcium | |
| Lysosomal- | LYST, | May be required for | Chediak-Higashi |
| trafficking | CHS | sorting endosomal | syndrome. A rare |
| regulator | | resident proteins | autosomal recessive |
| (Beige | | into late | disorder |
| homolog) | | multivesicular | characterized by |
| | | endosomes by a | hypopigmentation, |
| | | mechanism | severe immunologic |
| | | involving | deficiency, a |
| | | microtubules | bleeding tendency, |
| | | | neurologic |
| | | | abnormalities, |
| | | | abnormal |
| | | | intracellular |
| | | | transport to and |
| | | | from the lysosome, |
| | | | and giant inclusion |
| | | | bodies in a variety |
| | | | of cell types. Most |
| | | | patients die at an |
| | | | early age unless |
| | | | they receive an |
| | | | allogeneic |
| | | | hematopoietic stem |
| | | | cell transplant. |
| Serine- | ATM | Serine/threonine | Ataxia |
| protein | | protein kinase | telangiectasia (AT). |
| kinase | | which activates | A rare recessive |
| ATM (EC | | checkpoint signaling | disorder |
| 2.7.11.1) | | upon double strand | characterized by |
| (Ataxia | | breaks (DSBs), | progressive |
| telangi- | | apoptosis and | cerebellar ataxia, |
| ectasia | | genotoxic stresses | dilation of the blood |
| mutated) | | such as ionizing | vessels in the |
| (A-T | | ultraviolet A light | conjunctiva and |
| mutated) | | (UVA), thereby | eyeballs, |
| | | acting as a DNA | immunodeficiency, |
| | | damage sensor. | growth retardation |
| | | Recognizes the | and sexual |
| | | substrate consensus | immaturity. Patients |
| | | sequence [ST]-Q. | have a strong |
| | | Phosphorylates ‘Ser- | predisposition to |
| | | 139’ of histone | cancer; about 30% |
| | | variant H2AX at | of patients develop |
| | | double strand breaks | tumors, particularly |
| | | (DSBs), thereby | lymphomas and |
| | | regulating DNA | leukemias. Cells |
| | | damage response | from affected |
| | | mechanism. Also | individuals are |
| | | plays a role in pre-B | highly sensitive to |
| | | cell allelic exclusion, | damage by ionizing |
| | | a process leading to | radiation and |
| | | expression of a single | resistant to |
| | | immunoglobulin | inhibition of DNA |
| | | heavy chain allele to | synthesis following |
| | | enforce clonality | irradiation. |
| | | and monospecific | Defects in ATM |
| | | recognition by the | may contribute |
| | | B-cell antigen | to T-cell acute |
| | | receptor (BCR) | lymphoblastic |
| | | expressed on | leukemia (TALL) |
| | | individual B- | and T-prolymphocytic |
| | | lymphocytes. After | leukemia (TPLL). |
| | | the introduction of | TPLL is |
| | | DNA breaks by the | characterized by a |
| | | RAG complex on | high white blood |
| | | one immunoglobulin | cell count, with a |
| | | allele, acts by | predominance of |
| | | mediating a | prolymphocytes, |
| | | repositioning of the | marked |
| | | second allele to | splenomegaly, |
| | | pericentromeric | lymphadenopathy, |
| | | heterochromatin, | skin lesions and |
| | | preventing | serous effusion. The |
| | | accessibility to the | clinical course is |
| | | RAG complex and | highly aggressive, |
| | | recombination of | with poor response |
| | | the second allele. | to chemotherapy |
| | | Also involved in | and short survival |
| | | signal transduction | time. TPLL occurs |
| | | and cell cycle | both in adults as a |
| | | control. May | sporadic disease and |
| | | function as a tumor | in younger AT |
| | | suppressor. | patients. |
| | | Necessary for | Defects in ATM |
| | | activation of ABL1 | may contribute to B- |
| | | and SAPK. | cell chronic |
| | | Phosphorylates | lymphocytic |
| | | DYRK2, CHEK2, | leukemia (BCLL). |
| | | p53/TP53, | BCLL is the |
| | | FANCD2, | commonest form of |
| | | NFKBIA, BRCA1, | leukemia in the |
| | | CTIP, nibrin | elderly. It is |
| | | (NBN), TERF1, | characterized by the |
| | | RAD9, UBQLN4 | accumulation of |
| | | and DCLRE1C. | mature CD5+ B- |
| | | May play a role in | lymphocytes, |
| | | vesicle and/or | lymphadenopathy, |
| | | protein transport. | immunodeficiency |
| | | Could play a role in | and bone marrow |
| | | T-cell development, | failure. |
| | | gonad and | |
| | | neurological | |
| | | function. Plays a | |
| | | role in replication- | |
| | | dependent histone | |
| | | mRNA degradation. | |
| | | Binds DNA ends. | |
| | | Phosphorylation of | |
| | | DYRK2 in nucleus | |
| | | in response to | |
| | | genotoxic stress | |
| | | prevents its MDM2- | |
| | | mediated | |
| | | ubiquitination and | |
| | | subsequent | |
| | | proteasome | |
| | | degradation. | |
| | | Phosphorylates | |
| | | ATF2 which | |
| | | stimulates its | |
| | | function in DNA | |
| | | damage response. | |
| | | Phosphorylates | |
| | | ERCC6 which is | |
| | | essential for its | |
| | | chromatin | |
| | | remodeling activity | |
| | | at DNA double- | |
| | | strand breaks. | |
| Tuberin | TSC2 | In complex with | Tuberous sclerosis 2 |
| | | TSC1, this tumor | (TSC2). An |
| | | suppressor inhibits | autosomal dominant |
| | | the nutrient- | multi-system |
| | | mediated or growth | disorder that affects |
| | | factor-stimulated | especially the brain, |
| | | phosphorylation of | kidneys, heart, and |
| | | S6K1 and | skin. It is |
| | | EIF4EBP1 by | characterized by |
| | | negatively | hamartomas (benign |
| | | regulating mTORC1 | overgrowths |
| | | signaling. Acts as a | predominantly of a |
| | | GTPase-activating | cell or tissue type |
| | | protein (GAP) for | that occurs normally |
| | | the small GTPase | in the organ) and |
| | | RHEB, a direct | hamartias |
| | | activator of the | (developmental |
| | | protein kinase | abnormalities of |
| | | activity of | tissue combination). |
| | | mTORC1. May also | Clinical |
| | | play a role in | manifestations |
| | | microtubule- | include epilepsy, |
| | | mediated protein | learning difficulties, |
| | | transport (By | behavioral |
| | | similarity). Also | problems, and skin |
| | | stimulates the | lesions. Seizures can |
| | | intrinsic GTPase | be intractable and |
| | | activity of the Ras- | premature death can |
| | | related proteins | occur from a variety |
| | | RAP1A and RAB5. | of disease- |
| | | | associated causes. |

### Conclusions

These data are highly supportive of the concept that bone marrow stem cells (which are known to circulate in the blood stream) could contribute to skin integrity and orchestration of wound repair via donation of its secreted cargo proteins. Analysis of the bone marrow mesenchymal stem cell secretome from 4 healthy donors revealed novel secreted protein cargo. Stem cell therapy, while effective in many settings, carries a risk of graft-versus-host disease and malignant transformation, thus, understanding whether a secretome-only approach may provide beneficial protein factors to diseased skin is of high clinical significance. Many proteins traditionally thought of as intracellular proteins were detected in the secretome after isolation of extracellular vesicles with the secretome. This is a novel study that explored the commonalities among the secretome of healthy bone marrow donors and how this common protein cargo revealed several important structural and functional proteins relevant to skin homeostasis and disease.

A significant fraction of the common proteins are classified as membrane proteins, which suggests there is a significant presence of intercellular protein transport via extracellular vesicles. The most common biological process detected was “transport”, emphasizing the important role the bone marrow MSC secretome has in transporting important proteins to its intended recipient tissue and cells. Other important processes, such as regulation of transcription, the cell cycle, and DNA damage may help explain some of the beneficial effects seen in many previous studies of the effects of BM-MSCs on a variety of diseases, including acute and chronic wound healing.

Type VII collagen is present in the stratified squamous epithelial basement membrane and forms the anchoring fibrils that contribute to epithelial basement membrane organization and adherence by interacting with extracellular matrix proteins, such as type IV collagen. When absent in the skin, patients with dystrophic epidermolysis bullosa or epidermolysis bullosa pruriginosa develop severe blistering, resulting in widespread chronic wounds, scarring and increased risk of infections. Previous studies showed the potential beneficial effects of bone marrow transplants in patients with recessive dystrophic epidermolysis bullosa, in part due to regeneration of collagen VII present at the basement membrane in epidermolysis bullosa patients (Wagner J E, Ishida-Yamamoto A, McGrath J A, Hordinsky M, Keene D R, Woodley D T et al. Bone marrow transplantation for recessive dystrophic epidermolysis bullosa. N Engl J Med 2010; 363:629-39). It has been previously shown that type VII collagen co-purified with BM-MSC EVs (McBride J D, Rodriguez-Menocal L, Candanedo A, Guzman W, Garcia-Contreras M, Badiavas E V. Dual mechanism of type VII collagen transfer by bone marrow mesenchymal stem cell extracellular vesicles to recessive dystrophic epidermolysis bullosa fibroblasts. Biochimie 2018; 155:50-8). This study is novel in revealing that type VII collagen was present in the secretome of 4 healthy donors and co-purified with extracellular vesicles from all 4 donors. Further biochemical studies should elucidate whether the type VII collagen association with vesicles is via direct binding to lipid membrane, via a protein-binding partner, or other molecular forces (such as affinity among hydrophobic macromolecules).

Type IV collagen was also found in the BM-MSC secretome of all 4 donors. Type IV collagen is the major structural component of basement membranes—the lamina densa in the skin and the foundation of the glomerular basement membrane in the kidney—forming a meshwork together with laminins, proteoglycans and entactin/nidogen (Abreu-Velez A M, Howard M S. Collagen IV in Normal Skin and in Pathological Processes. N Am J Med Sci 2012; 4:1-8). When the skin undergoes deep injury, the basement membrane components, including the lamina densa and type IV collagen, must be regenerated in an organized fashion to prevent scarring. It has been shown that type IV collagen is induced by BM stem cells in animal models of genetic kidney disease (Alport disease) (Sugimoto H, Mundel T M, Sund M, Xie L, Cosgrove D, Kalluri R. Bone-marrow-derived stem cells repair basement membrane collagen defects and reverse genetic kidney disease. Proc Natl Acad Sci USA 2006; 103:7321-6). This study supports the concept that BM-MSCs produce type IV collagen which is a helpful substrate for cutaneous wound healing.

This study finds that plectin was detected in the secretome of 4 healthy BM-MSC donors. Plectin interlinks intermediate filaments with microtubules and microfilaments and anchors intermediate filaments to desmosomes or hemidesmosomes. Plectin binds muscle proteins such as actin to membrane complexes in muscle, and plays major role in the maintenance of myofiber integrity. When plectin is mutated, it results in forms of epidermolysis bullosa simplex with muscular dystrophy and/or pyloric atresia, and epidermolysis bullosa simplex, Ogna type (in which patients develop widespread blistering at the level of the hemidesmosome) (Pfendner E, Rouan F, Uitto J. Progress in epidermolysis bullosa: the phenotypic spectrum of plectin mutations. Exp Dermatol 2005; 14:241-9). This study is unique in demonstrating the potential of the BM-MSC secretome to help repair the skin in patients with plectin-deficient epidermolysis bullosa simplex subtypes.

Bullous pemphigoid antigen 1 (BPAG1/dystonin) is a cytoskeletal linker protein that acts as a connector between intermediate filaments, actin and microtubule cytoskeleton networks. A mutation in BPAG1 leads to epidermolysis bullosa simplex, autosomal recessive 2, characterized by localized blistering on the dorsal, lateral and plantar surfaces of the feet and trauma-induced blistering mainly occurring on the feet and ankles. Ultrastructural analysis of skin biopsy shows abnormal hemidesmosomes with poorly formed inner plaques (Groves R W, Liu L, Dopping-Hepenstal P J, Markus H S, Lovell P A, Ozoemena L et al. A homozygous nonsense mutation within the dystonin gene coding for the coiled-coil domain of the epithelial isoform of BPAG1 underlies a new subtype of autosomal recessive epidermolysis bullosa simplex. J Invest Dermatol 2010; 130:1551-7). This study finds that 4 healthy donors' BM-MSCs secreted BPAG1. This study supports that the secretome of BM-MSCs can ameliorate the autosomal recessive 2 subtype of epidermolysis bullosa simplex.

All 4 donors' BM-MSCs secreted epiplakin, which is a cytoskeletal linker protein that connects to intermediate filaments and controls their reorganization in response to stress, such as mechanical stress like wound healing (Jang S I, Kalinin A, Takahashi K, Marekov L N, Steinert P M. Characterization of human epiplakin: RNAi-mediated epiplakin depletion leads to the disruption of keratin and vimentin IF networks. J Cell Sci 2005; 118:781-93). Epiplakin is associated with the cellular motility machinery by slowing down keratinocyte migration and proliferation and accelerating keratin bundling in proliferating keratinocytes, thus contributing to tissue architecture. In response to cellular stress, epiplakin plays a role in keratin filament reorganization, probably by protecting keratin filaments against disruption. This study is novel in finding that BM-MSCs produce epiplakin.

Keratin 1 was detected in the secretome among all 4 donors. Keratins are a group of fibrous proteins that form structural frameworks for keratinocytes to make up the skin, hair, and nails. While production is typically attributed to the keratinocytes, in this study keratin 1 was detected in the secretome of BM-MSCs of all four donors. Keratin 1 partners with either keratin 9 or 10 to form heterodimer intermediate filaments, which then assemble into strong networks that provide tensile strength and resiliency to the skin, protecting it from external damage. While genetic mutations in keratin 1 are typically autosomal dominant and lead to epidermolytic hyperkeratosis, one can consider any damaged cutaneous tissue (skin, hair, nails) potentially in need of a fresh supply of keratin 1 (especially if the keratinocytes have been damaged in the skin). This study supports an important role of the BM-MSC secretome in providing a fresh supply of keratin 1 to support the skin during homeostasis, repair and regeneration.

E-cadherin is a calcium-dependent cell adhesion protein vital in keratinocyte-to-keratinocyte adhesion and has been known to be produced by bone marrow cells (Turel K R, Rao S G. Expression of the cell adhesion molecule E-cadherin by the human bone marrow stromal cells and its probable role in CD34(+) stem cell adhesion. Cell Biol Int 1998; 22:641-8). This study found that E-cadherin was detected in the extracellular vesicle purified BM-MSC secretome from 4 healthy donors. Given its role in adhesion of epithelial cells, it has been associated with a variety of disease pathologies. It has also been associated with stimulation of tumor angiogenesis and was found to localize to the exosome surface (Tang M K S, Yue P Y K, Ip P P, Huang R L, Lai H C, Cheung A N Y et al. Soluble E-cadherin promotes tumor angiogenesis and localizes to exosome surface. Nat Commun 2018; 9:2270).

A protein called hSPCA1 (encoded by the gene ATP2C1) was detected in the BM-MSC secretome of all 4 donors. This protein is an ATP-powered calcium pump to transfer calcium and manganese ions across membranes in the Golgi apparatus (Micaroni M, Giacchetti G, Plebani R, Xiao G G, Federici L. ATP2C1 gene mutations in Hailey-Hailey disease and possible roles of SPCA1 isoforms in membrane trafficking. Cell Death Dis 2016; 7:e2259). When defective, this leads to disruption of keratinocyte-to-keratinocyte adhesion (in part due to subsequent cadherin dysfunction), resulting in acantholysis pathologically and blistering clinically. Defective hSPCA1 production results in a disease called benign familial pemphigus (Hailey-Hailey). This study supports that the donor BM-MSC secretome would be effective to ameliorate the effects of benign familial pemphigus.

Lysosomal trafficking regulator (encoded by the gene LYST) was found in the secretome of all 4 BM-MSC donors. Lysosomal trafficking regulator appears to be required for sorting endosomal resident proteins into late multivesicular endosomes by a mechanism involving microtubules (Song Y, Dong Z, Luo S, Yang J, Lu Y, Gao B et al. Identification of a compound heterozygote in LYST gene: a case report on Chediak-Higashi syndrome. BMC Med Genet 2020; 21:4). When patients have a defect in lysosomal trafficking regulator, patients develop Chediak-Higashi syndrome, a rare autosomal recessive disorder characterized by hypopigmentation, severe immunologic deficiency, a bleeding tendency, neurologic abnormalities, abnormal intracellular transport to and from the lysosome, and giant inclusion bodies in a variety of cell types. Most patients die at an early age unless they receive an allogeneic hematopoietic stem cell transplant. This study supports that the beneficial effects of bone marrow transplants in these patients may be mediated, at least in part, by a circulating form of lysosomal trafficking regulator that may make its way into multiple recipient tissues.

Serine/threonine protein kinase ATM, which activates checkpoint signaling upon double strand breaks, apoptosis, and genotoxic stresses such as ionizing ultraviolet light, was found in the secretome of all 4 BM-MSC donors. This kinase is also thought to be involved in signal transduction and cell cycle control and may function as a tumor suppressor. When mutated, patients develop ataxia-telangiectasia, a rare recessive disorder characterized by progressive cerebellar ataxia, dilation of the blood vessels in the conjunctiva, immunodeficiency, growth retardation and sexual immaturity. Patients have a strong predisposition to cancer; about 30% of patients develop tumors, particularly lymphomas and leukemias. This study supports that BM-MSCs would ameliorate the phenotype of ataxia-telangiectasia.

Tuberin is a protein encoded by the gene TSC-2, which, in complex with TSC1, this tumor suppressor inhibits the nutrient-mediated or growth factor-stimulated phosphorylation of growth factors by negatively regulating mTORC1 signaling (Henske E P, Jozwiak S, Kingswood J C, Sampson J R, Thiele E A. Tuberous sclerosis complex. Nat Rev Dis Primers 2016; 2:16035). When mutated, it leads to a phenotype of tuberous sclerosis complex, which is an autosomal dominant multi-system disorder that affects the brain, kidneys, heart, and skin. It is characterized by hamartomas (benign overgrowths predominantly of a cell or tissue type that occurs normally in the organ). Clinical manifestations include epilepsy, learning difficulties, behavioral problems, and skin lesions. Seizures can be intractable and premature death can occur from a variety of disease-associated causes. This study finds that tuberin is expressed in the secretome of healthy BM-MSC donors.

This study supports that the cargo proteins we detected in all 4 bone marrow donors could be isolated from the BM-MSC secretome and be delivered to recipient tissues, especially in those patients lacking functional proteins responsible for disease phenotypes. This study also supports that the secretome of BM-MSCs is beneficial for amelioration of a variety of dermatologic diseases.

This study identified proteins in the secretome of healthy donors of BM-MSCs. Some of these proteins had not been previously associated as secreted by any cell type. BM-MSC extracellular vesicles may help transfer important intracellular proteins between cells, explaining the benefit seen in dermatologic diseases, such as epidermolysis bullosa, after bone marrow transplants. This study supports that the secretome of BM-MSCs, rather than the cells themselves, are efficacious in ameliorate various aforementioned dermatologic diseases.

1. A method of treating:
(a) a condition selected from the group consisting of epidermolysis bullosa pruriginosa; epidermolysis bullosa acquisita; epidermolysis bullosa dystrophica, pretibial type; epidermolysis bullosa dystrophica, bart type; nonsyndromic congenital nail disorder-8; epidermolysis bullosa dystrophica, with subcorneal cleavage; and transient bullous dermolysis of the newborn in a subject in need thereof comprising administering a therapeutically effective amount of microvesicles, wherein the microvesicles comprise type VII collagen; or
(b) Alport syndrome 2, autosomal recessive in a subject in need thereof comprising administering a therapeutically effective amount of microvesicles, wherein the microvesicles comprise type IV collagen; or
(c) a condition selected from the group consisting of epidermolysis bullosa simplex with muscular dystrophy; epidermolysis bullosa simplex with pyloric atresia; epidermolysis bullosa, ogna type; epidermolysis bullosa simplex with nail dystrophy; and muscular dystrophy, limb-girdle, autosomal recessive 17 in a subject in need thereof comprising administering a therapeutically effective amount of microvesicles, wherein the microvesicles comprise plectin; or
(d) a condition selected from the group consisting of epidermolysis bullosa simplex, autosomal recessive 2 and neuropathy, hereditary sensory and autonomic, 6 in a subject in need thereof comprising administering a therapeutically effective amount of microvesicles, wherein the microvesicles comprise bullous pemphigoid antigen 1; or
(e) epidermolytic hyperkeratosis in a subject in need thereof comprising administering a therapeutically effective amount of microvesicles, wherein the microvesicles comprise keratin 1; or
(f) benign familial pemphigus in a subject in need thereof comprising administering a therapeutically effective amount of microvesicles, wherein the microvesicles comprise hSPCA1; or
(g) Chediak-Higashi syndrome in a subject in need thereof comprising administering a therapeutically effective amount of microvesicles, wherein the microvesicles comprise lysosomal trafficking regulator; or
(h) a condition selected from the group consisting of ataxia telangiectasia syndrome; T-cell acute lymphoblastic leukemia; T-cell prolymphocytic leukemia; and B-cell chronic lymphocytic leukemia in a subject in need thereof comprising administering a therapeutically effective amount of microvesicles, wherein the microvesicles comprise serine-protein kinase ATM; or
(i) tuberous sclerosis 2 in a subject in need thereof comprising administering a therapeutically effective amount of microvesicles, wherein the microvesicles comprise tuberin; or
(j) diabetic foot ulcers in a subject in need thereof comprising administering a therapeutically effective amount of microvesicles, wherein the microvesicles comprise FOXM1A.

2. The method of claim 1, wherein:
(a) the condition is epidermolysis bullosa pruriginosa, wherein the microvesicles alleviate or reduce one or more symptoms of epidermolysis bullosa pruriginosa in the subject, and wherein the symptoms of epidermolysis bullosa pruriginosa are selected from the group consisting of pruritus, blisters, chronic wounds, scar formation, increased risk of skin infections, milia, skin fragility, nail dystrophy, lichenified plaques, albopapuloid lesions, and excoriated prurigo nodules; or
(b) the condition is epidermolysis bullosa acquisita, wherein the microvesicles alleviate or reduce one or more symptoms of epidermolysis bullosa acquisita in the subject, and wherein the symptoms of epidermolysis bullosa acquisita are selected from the group consisting of blistering, milia, wound healing with significant scarring, skin itching, and skin redness; or
(c) the condition is epidermolysis bullosa dystrophica, pretibial type, wherein the microvesicles alleviate or reduce one or more symptoms of epidermolysis bullosa dystrophica, pretibial type in the subject, and the symptoms of epidermolysis bullosa dystrophica, pretibial type are selected from the group consisting of pretibial blisters, prurigo-like hyperkeratotic lesions, nail dystrophy, albopapuloid skin lesions, and hypertrophic scars; or
(d) wherein the condition is epidermolysis bullosa dystrophica, bart type, wherein the microvesicles alleviate or reduce one or more symptoms of epidermolysis bullosa dystrophica, bart type in the subject, and wherein the symptoms of epidermolysis bullosa dystrophica, bart type are selected from the group consisting of congenital localized absence of skin, skin fragility, and deformity of the nails; or
(e) the condition is nonsyndromic congenital nail disorder-8, wherein the microvesicles alleviate or reduce one or more symptoms of nonsyndromic congenital nail disorder-8 in the subject, and wherein the symptoms of nonsyndromic congenital nail disorder-8 comprise toenail dystrophy and/or the nail plate being buried in the nail bed with a deformed and narrow free edge; or
(f) the condition is epidermolysis bullosa dystrophica, with subcorneal cleavage, wherein the microvesicles alleviate or reduce one or more symptoms of epidermolysis bullosa dystrophica, with subcorneal cleavage in the subject, and wherein the symptoms of epidermolysis bullosa dystrophica, with subcorneal cleavage are selected from the group consisting of blisters, milia, atrophic scarring, and nail dystrophy; or
(g) the condition is transient bullous dermolysis of the newborn, wherein the microvesicles alleviate or reduce one or more symptoms of transient bullous dermolysis of the newborn in the subject, and wherein the symptoms of transient bullous dermolysis of the newborn are selected from the group consisting of sub-epidermal blisters, reduced or abnormal anchoring fibrils at the dermo-epidermal junction, and electron-dense inclusions in keratinocytes.

3-22. (canceled)

23. The method of claim 1, wherein the subject:
(a) has a mutation in the COL7A1 gene, and wherein the microvesicles deliver collagen VII protein to the cells of the subject; or
(b) has a mutation in the COL4A4 gene, and wherein the microvesicles deliver type IV collagen protein to the cells of the subject; or
(c) has a mutation in the PLEC1 gene, and wherein the microvesicles deliver plectin protein to the cells of the subject; or
(d) has a mutation in the BPAG1 gene, and wherein the microvesicles deliver bullous pemphigoid antigen 1 protein to the cells of the subject; or
(e) has a mutation in the KRT1 gene, and wherein the microvesicles deliver keratin 1 protein to the cells of the subject; or
(f) has a mutation in the ATP2C1 gene, and wherein the microvesicles deliver hSPCA1 protein to the cells of the subject; or
(g) has a mutation in the LYST gene, and wherein the microvesicles deliver lysosomal trafficking regulator protein to the cells of the subject; or
(h) has a mutation in the ATM gene, and wherein the microvesicles deliver serine-protein kinase ATM protein to the cells of the subject; or
(i) has a mutation in the TSC2 gene, and wherein the microvesicles deliver tuberin protein to the cells of the subject; or
(j) has a mutation in the FOXM1A gene, and wherein the microvesicles deliver FOXM1A protein to the cells of the subject.

24. (canceled)

25. (canceled)

26. The method of claim 1, wherein the microvesicles alleviate or reduce one or more symptoms of Alport syndrome 2, autosomal recessive in the subject, and wherein the symptoms of Alport syndrome 2, autosomal recessive are selected from the group consisting of glomerulonephritis, glomerular basement membrane defects, renal failure, sensorineural deafness, lenticonous, macular flecks, and hematuria.

27-30. (canceled)

31. The method of claim 1, wherein:
(a) the condition is epidermolysis bullosa simplex with muscular dystrophy, wherein the microvesicles alleviate or reduce one or more symptoms of epidermolysis bullosa simplex with muscular dystrophy in the subject, and wherein the symptoms of epidermolysis bullosa simplex with muscular dystrophy are selected from the group consisting of hemorrhagic blisters, blister formation at the level of the hemidesmosome, nail dystrophy, palmoplantar keratoderma, and erosions of the skin and oral mucosae; or
(b) the condition is epidermolysis bullosa simplex with pyloric atresia, wherein the microvesicles alleviate or reduce one or more symptoms of epidermolysis bullosa simplex with pyloric atresia in the subject, and wherein the symptoms of epidermolysis bullosa simplex with pyloric atresia are selected from the group consisting of blistering, skin fragility, milia, nail dystrophy, scarring alopecia, and hypotrichosis; or
(c) the condition is epidermolysis bullosa, ogna type, wherein the microvesicles alleviate or reduce one or more symptoms of epidermolysis bullosa, ogna type in the subject, and wherein the symptoms of epidermolysis bullosa, ogna type are selected from the group consisting of skin bruising, skin fragility, blistering, and abnormal hemidesmosome intracellular attachment plates; or
(d) the condition is epidermolysis bullosa simplex with nail dystrophy, the microvesicles alleviate or reduce one or more symptoms of epidermolysis bullosa simplex with nail dystrophy in the subject, and wherein the symptoms of epidermolysis bullosa simplex with nail dystrophy comprise skin blistering and/or nail dystrophy; or
(e) the condition is muscular dystrophy, limb-girdle, autosomal recessive 17, wherein the microvesicles alleviate or reduce one or more symptoms of muscular dystrophy, limb-girdle, autosomal recessive 17 in the subject, and wherein the symptoms of muscular dystrophy, limb-girdle, autosomal recessive 17 are selected from the group consisting of proximal muscle weakness, weakness of the hip and shoulder girdles, prominent asymmetrical quadriceps femoris atrophy, and biceps brachii atrophy.

32-48. (canceled)

49. The method of claim 1, wherein the condition is epidermolysis bullosa simplex, autosomal recessive 2, wherein the microvesicles alleviate or reduce one or more symptoms of epidermolysis bullosa simplex, autosomal recessive 2 in the subject, and wherein the symptoms of epidermolysis bullosa simplex, autosomal recessive 2 are selected from the group consisting of blistering on the dorsal, lateral and plantar surfaces of the feet, trauma-induced blistering on the feet and ankles, and abnormal hemidesmosomes with poorly formed inner plaques.

50. (canceled)

51. (canceled)

52. The method of claim 1, wherein the condition is neuropathy, hereditary sensory and autonomic, 6, wherein the microvesicles alleviate or reduce one or more symptoms of neuropathy, hereditary sensory and autonomic in the subject, and wherein the symptoms of neuropathy, hereditary sensory and autonomic are selected from the group consisting of degeneration of dorsal root and autonomic ganglion cells, sensory abnormalities, and autonomic abnormalities.

56-57. (canceled)

58. The method of claim 1, wherein the microvesicles alleviate or reduce one or more symptoms of epidermolytic hyperkeratosis in the subject, and wherein the symptoms of epidermolytic hyperkeratosis are selected from the group consisting of intraepidermal blistering, thickening of the stratum corneum, pigmentation of the skin and erosions at sites of trauma, and erythroderma.

59-62. (canceled)

63. The method of claim 1, wherein the microvesicles alleviate or reduce one or more symptoms of benign familial pemphigus in the subject, and wherein the symptoms of benign familial pemphigus are selected from the group consisting of blisters, erosions of the skin, rash, cracked skin, and acantholysis.

64-67. (canceled)

68. The method of claim 1, wherein the microvesicles alleviate or reduce one or more symptoms of Chediak-Higashi syndrome in the subject, and wherein the symptoms of Chediak-Higashi syndrome are selected from the group consisting of hypopigmentation, severe immunologic deficiency, bleeding tendency, neurologic abnormalities, abnormal intracellular transport to and from the lysosome, and giant inclusion bodies in a variety of cell types.

69-72. (canceled)

73. The method of claim 1, wherein:
(a) the condition is ataxia telangiectasia syndrome, wherein the microvesicles alleviate or reduce one or more symptoms of ataxia telangiectasia syndrome in the subject, and wherein the symptoms of ataxia telangiectasia syndrome are selected from the group consisting of progressive cerebellar ataxia, dilation of the blood vessels in the conjunctiva and eyeballs, immunodeficiency, growth retardation, and sexual immaturity; or
(b) the condition is T-cell acute lymphoblastic leukemia, wherein the microvesicles alleviate or reduce one or more symptoms of T-cell acute lymphoblastic leukemia in the subject, and wherein the symptoms of T-cell acute lymphoblastic leukemia are selected from the group consisting of anemia, frequent infections due to the lack of normal white blood cells, frequent infections, fever, purpura, and nosebleeds and bleeding gums due to lack of platelets; or
(c) the condition is T-cell prolymphocytic leukemia, wherein the microvesicles alleviate or reduce one or more symptoms of T-cell prolymphocytic leukemia, and wherein the symptoms of T-cell prolymphocytic leukemia are selected from the group consisting of a high white blood cell count, a predominance of prolymphocytes, marked splenomegaly, lymphadenopathy, skin lesions, and serous effusion; or
(d) the condition is B-cell chronic lymphocytic leukemia, wherein the microvesicles alleviate or reduce one or more symptoms of B-cell chronic lymphocytic leukemia in the subject, and wherein the symptoms of B-cell chronic lymphocytic leukemia are selected from the group consisting of accumulation of mature CD5+B-lymphocytes, lymphadenopathy, immunodeficiency, and bone marrow failure.

74-87. (canceled)

88. The method of claim 1, wherein the microvesicles alleviate or reduce one or more symptoms of tuberous sclerosis 2 in the subject, and wherein the symptoms of tuberous sclerosis 2 are selected from the group consisting of hamartomas, hamartias, epilepsy, learning difficulties, behavioral problems, and skin lesions.

89-92. (canceled)

93. The method of claim 1, wherein the microvesicles alleviate or reduce one or more symptoms of diabetic foot ulcers in the subject, wherein the symptoms of diabetic foot ulcers comprise open sores or wounds on the foot of the subject.

94-96. (canceled)

97. The method of claim 1, wherein the microvesicles are derived from mesenchymal stem cells.

98. The method of claim 1, wherein the microvesicles are derived from mesenchymal stem cells, and wherein the mesenchymal stem cells are bone marrow mesenchymal stem cells.

99. The method of claim 1, wherein the microvesicles are obtained from a biological fluid and precipitated from the biological fluid using polyethylene glycol.

100. The method of claim 1, wherein the microvesicles are administered to the skin and/or nails of the subject.

101. The method of claim 1, wherein the microvesicles are administered via transplanted mesenchymal stem cells.

102. A composition comprising microvesicles derived from mesenchymal stem cells wherein the microvesicles comprise at least one active agent comprising type VII collagen, type IV collagen, plectin, bullous pemphigoid antigen 1, keratin 1, hSPCA1, serine-protein kinase ATM, tuberin, FOXM1A, or mixtures thereof.

103. The composition of claim 102 wherein the mesenchymal stem cells are bone marrow-derived mesenchymal stem cells.
